(19) United States
(12) Patent Application Publication
(10) Pub. No.: US 2012/0276066 A1
(43) Pub. Date: Nov. 1, 2012

(54) Peptide Linked Cell Matrix Materials for Stem Cells and Methods of Using the Same

(75) Inventors: Jan Engelsen Brinchmann, Oslo (NO); Katrine Bjornebek Fronsdal, Oslo (NO); Jan Egil Melvik, Oslo (NO)

(73) Assignee: FMC CORPORATION, Philadelphia, PA (US)

(21) Appl. No.: 13/494,623

(22) Filed: Jun. 12, 2012

Related U.S. Application Data
(62) Division of application No. 12/663,945, filed on May 5, 2010.
(60) Provisional application No. 60/943,821, filed on Jun. 13, 2007.
(60) Provisional application No. 61/013,145, filed on Dec. 12, 2007.

(51) Int. Cl. A61K 35/12; A61P 25/28; A61P 9/10; A61P 35/00; A61P 19/10; A61P 9/00; A61P 29/00; A61P 1/00; A61P 25/16; A61P 19/02; A61P 25/00; A61P 3/10

(52) U.S. Cl. 424/93.7

(57) ABSTRACT

Biostructures that comprises modified alginates entrapping one or more stem cells are discloses. The modified alginates comprise at least one alginate chain section to which is bonded by covalent bonding at least one cell attachment peptide. Pluralities of stem cells are also disclosed. Methods of preventing death of stem cells and cells differentiated there from are disclosed. Methods of preparing a plurality of stem cells are disclosed. Methods of treating an individual who has a degenerative disease, such as a neurological disorder, or injury involving nerve damage by administering stem cells to said individual are disclosed.

### FIELD OF THE INVENTION

The present invention relates to stem cells, compositions comprising stem cells, methods of preparing stem cells and compositions comprising stem cells using cell adhesion peptides and methods of using stem cells and compositions comprising stem cells.

### BACKGROUND OF THE INVENTION

Recognizing the micro-environmental property that affect cellular gene expression, phenotype and function is important for the better understanding of cells, as well as to provide better approaches to engineer artificial tissues for medical applications. In their normal environment mammalian cells are embedded within a complex and dynamic microenvironment consisting of the surrounding extracellular matrix, growth factors, and cytokines, as well as neighbouring cells. Cell adhesion to the extracellular matrix scaffolding involves physical connection to the extracellular matrix proteins through specific cell surface receptors. Of these, integrins are the major transmembrane receptors responsible for connecting the intracellular cytoskeleton to the extracellular matrix. The adhesive processes trigger a cascade of intracellular signalling events that may lead to changes in cellular behaviours, such as growth, migration, and differentiation. Since materials derived from natural extracellular matrix, such as collagen, provide natural adhesive ligands that promote cell attachment through integrins, they have been a starting point for engineering biomaterials for tissue engineering. However, a major drawback of collagen and other biological materials is that our ability to control their chemical and physical properties is limited. The discovery of short peptide sequences that initiate cellular adhesion, such as arginine-glycine-aspartic acid (RGD), however, has allowed development of polymers onto which these adhesive peptides can be conjugated.

One group of polymers that have very promising properties in this respect are alginates. Alginates are hydrophilic marine biopolymers with the unique ability to form heat-stable gels that can develop and set at physiologically relevant temperatures. Alginates are a family of non-branched binary copolymers of 1-4 glycosidically linked β-D-mannuronic acid (M) and α-L-guluronic acid (G) residues. The relative amount of the two uronic acid monomers and their sequential arrangement along the polymer chain vary widely, depending on the origin of the alginate. Alginate is the structural polymer in marine brown algae and is also produced by certain bacteria. It has been demonstrated that peptides like RGD may be covalently linked to alginate, and that gel structures made of alginate may support cell adhesion.

Another critical factor in tissue engineering is the source of cells to be utilized. It has been found that immature cells are able to multiply to a higher degree in vitro than fully differentiated cells of specialized tissues. In contrast to the in vitro multiplication of fully differentiated cells, such immature or progenitor cells can be induced to differentiate and function after several generations in vitro. They also appear to have the ability to differentiate into many of the specialized cells found within specific tissues as a function of the environment in which they are placed. Therefore, stem cells may be the cell of choice for tissue engineering.

Current technology allows cultivation of stem cells in vitro as monolayer cultures. However, in order to differentiate stem cells into a specific phenotype, there is a demand for biocompatible matrixes giving optimal conditions for cell function, proliferation and differentiation in a three dimensional environment.

### SUMMARY OF THE INVENTION

The present invention relates to biostructures that comprises modified alginates entrapping one or more stem cells. The modified alginates comprise at least one alginate chain section to which is bonded by covalent bonding at least one cell attachment peptide.

The present invention also relates to pluralities of stem cells which have been isolated from such biostructures.

The present invention further relates to methods of inducing changes in gene expression by stem cells and cells differentiated there from within a three dimensional biostructure. The three dimensional biostructure comprises a modified alginate comprising at least one alginate chain section to which is bonded by covalent bonding at least one cell attachment peptide. The method comprises the step of entrapping stem cells and cells differentiated there from within the biostructure.

The present invention also relates to methods of preparing a plurality of stem cells. The methods comprise the steps of: obtaining one or more stem cells from a donor, maintaining stem cells obtained from a donor under conditions in which the stem cells grow and proliferate as a monolayer. The stem cells are then entrapped in a biostructure comprising a modified alginate that comprises at least one alginate chain section to which is bonded by covalent bonding at least one cell attachment peptide and then isolated from said biostructure.

The present invention additionally relates to a plurality of stem cells prepared by such methods.

The present invention also relates to methods of treating an individual who has a degenerative disease, such as a neurological disorder, or injury involving nerve damage by administering to said individual such stem cells. The method comprises the steps of culturing stem cells in a biostructure comprising a modified alginate that comprises at least one alginate chain section to which is bonded by covalent bonding at least one cell attachment peptide under conditions in which the stems cells proliferate and then administering the stem cells to an individual who has a neurological disorder or injury involving nerve damage in an amount effective and at a site effective to provide a therapeutic benefit to the individual.

### BRIEF DESCRIPTION OF THE FIGURES

FIG. 1 shows data of the fraction of dead fat derived stem cells at different times after entrapment in alginate beads made of alginate with or without covalently linked RGD sequences. The fraction of dead cells were also recorded in alginate beads with a 10 fold increased cell density (closed symbols). Standard error of the mean are indicated when exceeding the symbols.

FIG. 2 shows data of the fraction of dead bone marrow derived stem cells at different times after entrapment in alginate beads made of alginate with or without covalently linked RGD sequences. The fraction of dead cells were also recorded in alginate beads with a 10 fold increased cell density (closed symbols). Standard error of the mean are indicated when exceeding the symbols.

FIG. 3 shows data from two parametric flow cytometric recordings of bone marrow stem cells stained with BrdU (FL1) and propidium iodide (FL2). The gated regions (R2) show the fraction of cells with sub G1 DNA-content (non-viable cells).

FIG. 4, panel A shows a photograph of stem cells taken immediately after prospective isolation form source material. Before attachment and spreading, the uncultured AT-MSC were small and round. FIG. 4, panel B shows a photograph of stem cells taken after in vitro culture in 2D in monolayer. The AT-MSC adopted a spindle-shaped morphology. FIG. 4, panel C top panel, left and right shows photographs of stem cells entrapped in regular alginate. The MSC regain a spherical morphology, but a number of cells are dead on day 7 (FIG. 4, panel C top, middle panel, same as left panel but with fluorescent light in stead of white light). FIG. 4, panel C bottom panel, right shows stem cells in RGD alginate. The cells can be seen to have extensions protruding from the cell body, and the proportion of dead cell day 7 is much lower (FIG. 4, panel C bottom, middle panel, fluorescent light). The proportion of dead cells in regular alginate was increasing throughout 21 days in 3D culture (FIG. 4, panel D, grey bars), while the proportion of dead cells in RGD alginate was low and quite stable throughout this culture period (FIG. 4, panel D, black bars). The total number of live and dead cells did not change in the course of culture in regular alginate (grey bars) or RGD alginate (black bars) for AT-MSV (FIG. 4, panel E, left panel) or BM-MSC (FIG. 4, panel E, right panel). Slightly different numbers of cells were seeded per bead for AT-MSC and BM-MSC.

FIG. 5 shows death of MSC in regular alginate is due by PCD. FIG. 5, panel A shows the results of a TUNEL assay performed on AT-MSC on day 7 of culture in regular alginate, showing the same cells in fluorescent light (top) and white light (bottom). The amount of PCD on day 7 was quantified by gating on the subG1 population in BrdU assays performed on cells in monolayer culture (FIG. 5, panel B, top), regular alginate (FIG. 5, panel B, middle) and RGD alginate (FIG. 5, panel B, bottom) for AT-MSC (FIG. 5, panel B, left) and BM-MSC (FIG. 5, panel B, right). The numbers are the percentage of cells in the subG 1 gate. Results from single experiments are representative for two experiments for each cell population. The proportion of live cells in S-phase of cell cycle was quantified by removing the subG1 population from the BrdU assays, and then gating on cells in S-phase (FIG. 5, panel C). The numbers are the percentage of live cells in S-phase. 3H thymidine incorporation assay (FIG. 5, panel D) for AT-MSC from five donors (top) and BM-MSC from three donors (bottom) comparing cells in monolayer cultures and cells cultured in regular alginate or RGD-alginate for 7 days. Freshly isolated T-cells were used as experimental controls for cells that were unlikely to incorporate 3H thymidine.

FIG. 6 shows flow cytometric analysis of the expression of integrin monomers on cells cultured in monolayer (top), regular alginate (middle) and RGD alginate (bottom panels).

### DESCRIPTION OF EMBODIMENTS OF THE INVENTION

Cell attachment peptides covalently linked to alginates are supportive for stem cells and cells differentiated therefrom as cell matrix materials. Stem cells cultivated in alginate beads that have covalently linked cell attachment peptides undergo changes in gene expression profile compared to stem cells cultivated in beads made of alginates without covalently linked cell attachment peptides. In some experiments, cell attachment peptides covalently linked to alginates have been observed to be aid in maintaining cell survival.

Gene expression changes when stem cells obtained from source material are cultivated as a monolayer. Further, when stem cells cultivated as a monolayer are removed from the monolayer and cultured in alginate beads that have covalently linked cell attachment peptides, the gene expression profile changes further. Stem cells passaged through monolayers and cultured in alginate beads that have covalently linked cell attachment peptides have different expression profiles from the expression profile of the uncultured stem cells obtained from source material. Without being bound by any theory, it is believed that as the alginates having cell attachment peptides covalently linked thereto support stem cell adhesion, promote changes in gene expression, and may prevent cells from undergoing apoptosis (or other forms of cell death). Such alginate having cell attachment peptides covalently linked thereto may thus be used in different biostructures as a way to promote changes in gene expression and in some instances maintain stem cell survival. Such alginate biostructures include alginate gels, but may also include foam or fibre structures and others.

The discovery that the alginates of the invention change expression profiles of stem cells may be used in tissue engineering applications as well as in the culturing of stem cells to expand and maintain populations of cells for use in various methods including subsequent administration into an individual.

One aspect of the present invention is directed to a method for passaging stem cell within a three dimensional biostructure comprising cell adhesion peptide-coupled alginates, e.g., RGD peptides covalently linked to alginate and biostructures made therefrom comprising viable stem cells in a gel. Suitable biostructures of the invention include foam, film, gels, beads, sponges, felt, fibers and combinations thereof.

One property of alginate gel structures containing cells or other constituents is that the entrapped material may be released after dissolving the gel. Alginate having cell attachment peptides covalently linked thereto gels may be dissolved thereby releasing the entrapped stem cells. This may be performed by using cation binding agents like citrates, lactates or phosphates. This holds a very useful property as the stem cells (and cells differentiated there from) may be removed from the gel structures and their properties may be tested in relation to a specific application. The cells may then be tested for the expression of specific genes, surface expression or others. Also the released stem cells (and cells differentiated there from) may be further cultivated as a monolayer culture or used in a three dimensional structure like an alginate gel or other for use as a tissue construct, as a cell encapsulation system or others.

Another aspect of the invention provides that stem cells may be obtained from sources, cultured as monolayers to promote cell proliferation and to obtain expanded numbers, then entrapped and maintained in biostructures comprising cell adhesion peptide-coupled alginates after which the cells are isolated from the biostructures and a population of stem cells is obtained with a gene expression pattern that is different from the monolayer expanded population. Such difference in gene expression pattern makes the population of stem cells particularly useful for administration to individuals and the treatment of diseases such as degenerative diseases.

When cells cultured as monolayers are entrapped within biostructures comprising cell adhesion peptide-coupled alginates, the cells change in morphology and gene expression. The cells become generally spherical and among the changes in gene expression, expression of genes encoding integrins changes. Cells are maintained as entrapped in biostructures for a time sufficient for gene expression to change from the expression profile exhibited by cells cultured as a monolayer to the stable gene expression profile exhibited by cells maintained in biostructure. In some embodiments, cells are maintained as entrapped in biostructures for at least 3 hours prior to removal of biostructure. In some embodiments, cells are maintained as entrapped in biostructures for less than 6 hours prior to removal of biostructure. In some embodiments, cells are maintained as entrapped in biostructures for at least 6 hours prior to removal of biostructure. In some embodiments, cells are maintained as entrapped in biostructures for less than 9 hours prior to removal of biostructure. In some embodiments, cells are maintained as entrapped in biostructures for at least 9 hours prior to removal of biostructure. In some embodiments, cells are maintained as entrapped in biostructures for less than 12 hours prior to removal of biostructure. In some embodiments, cells are maintained as entrapped in biostructures for at least 12 hours prior to removal of biostructure. In some embodiments, cells are maintained as entrapped in biostructures for less than 18 hours prior to removal of biostructure. In some embodiments, cells are maintained as entrapped in biostructures for at least 18 hours prior to removal of biostructure. In some embodiments, cells are maintained as entrapped in biostructures for less than 24 hours prior to removal of biostructure. In some embodiments, cells are maintained as entrapped in biostructures for at least 24 hours prior to removal of biostructure. In some embodiments, cells are maintained as entrapped in biostructures for less than 36 hours prior to removal of biostructure. In some embodiments, cells are maintained as entrapped in biostructures for at least 36 hours prior to removal of biostructure. In some embodiments, cells are maintained as entrapped in biostructures for less than 48 hours prior to removal of biostructure. In some embodiments, cells are maintained as entrapped in biostructures for at least 48 hours prior to removal of biostructure. In some embodiments, cells are maintained as entrapped in biostructures for less than 72 hours prior to removal of biostructure. In some embodiments, cells are maintained as entrapped in biostructures for at least 72 hours prior to removal of biostructure. In some embodiments, cells are maintained as entrapped in biostructures for at least 4 days prior to removal of biostructure. In some embodiments, cells are maintained as entrapped in biostructures for at least 5 days hours prior to removal of biostructure. In some embodiments, cells are maintained as entrapped in biostructures for at least 6 days prior to removal of biostructure. In some embodiments, cells are maintained as entrapped in biostructures for at least 1 week prior to removal of biostructure. In some embodiments, cells are maintained as entrapped in biostructures for up to 2 weeks prior to removal of biostructure. In some embodiments, cells are maintained as entrapped in biostructures for up to 3 weeks prior to removal of biostructure. In some embodiments, cells are maintained as entrapped in biostructures for up to 4 weeks prior to removal of biostructure.

Another aspect of the invention provides that stem cells may be obtained from sources and entrapped and cultured in biostructures comprising cell adhesion peptide-coupled alginates after which the cells are isolated from the biostructures and a population of stem cells is obtained with a gene expression pattern that is different from the monolayer expanded population. In such embodiment, the stems cells chosen are preferably those which are capable of proliferation under such conditions such as stem cells derived from adipose tissue. Such stem cells may be useful for administration to individuals and the treatment of diseases such as degenerative diseases.

According to some embodiments, stem cells are cultured in alginate matrices made from alginate polymers that comprise alginate polymers covalently linked to cell attachment peptides such as but not limited to those having the RGD motif. Such stem cells cultured in such matrices may be useful in the treatment of neurological disorders, such as for example Parkinson disease, HD (Huntington's disease), stroke, mucopolysaccharidosis and MS (Multiple Sclerosis), and in the treatment of injuries involving nerve damage such as spinal injuries. Such stem cells may be implanted into the patient such as in the brain, spinal column or other appropriate site where they can impart a therapeutic effect.

The stem cells of the invention may be delivered to the patient by any mode of delivery such as implantation at the site where therapeutic effect is desirable, or systemically. Modes of administration include direct injection or implantation. The stem cells of the invention may be delivered as part of a composition or device or as encapsulated or unencapsulated cells. In some embodiments, the stem cells are delivered intravenously, intrathecally, subcutaneously, directly into tissue of an organ, directly into spaces and cavities such as synovial cavities and spinal columns or nerve pathways. The intravenous administration of the stem cells of the invention may be less likely to result in accumulation of stem cells in the lung, a pattern which is observed when stem cells are administered intravenously directly after culturing as a monolayer.

The stem cells of the present invention may be useful in the treatment of degenerative disease, i.e a disease in which the function or structure of the affected tissues or organs progressively deteriorates over time. Examples of degenerative diseases include: Alzheimer's Disease; Amyotrophic Lateral Sclerosis (ALS), i.e., Lou Gehrig's Disease; Atherosclerosis; Cancer; Diabetes, Heart Disease; Huntington's disease (HD); Inflammatory Bowel Disease (IBD); mucopolysaccharidosis; Multiple Sclerosis (MS); Norrie disease; Parkinson's Disease; Prostatitis; Osteoarthritis; Osteoporosis; Shy-Drager syndrome; and Stroke.

Any stem cells may be used. In some embodiments, stem cells may be mesenchymal stem cells such as those derived from fat or bone marrow. In some embodiments, the stem cells are autologous. That is, they are derived from the individual into whom they and their progeny will be implanted.

U.S. Pat. Nos. 4,988,621, 4,792,525, 5,965,997, 4,879,237, 4,789,734 and 6,642,363, which are incorporated herein by reference, disclose numerous examples. Suitable peptides include, but are not limited to, peptides having about 10 amino acids or less. In some embodiments, cell attachment peptides comprise RGD, YIGSR (SEQ ID NO:1), IKVAV (SEQ ID NO:2), REDV (SEQ ID NO:3), DGEA (SEQ ID NO:4), VGVAPG (SEQ ID NO:5), GRGDS (SEQ ID NO:6), LDV, RGDV (SEQ ID NO:7), PDSGR (SEQ ID NO:8), RYVVLPR (SEQ ID NO:9), LGTIPG (SEQ ID NO:10), LAG, RGDS (SEQ ID NO:11), RGDF (SEQ ID NO:12), HHLGGALQAGDV (SEQ ID NO:13), VTCG (SEQ ID NO:14), SDGD (SEQ ID NO:15), GREDVY (SEQ ID NO:16), GRGDY (SEQ ID NO:17), GRGDSP (SEQ ID NO:18), VAPG (SEQ ID NO:19), GGGGRGDSP (SEQ ID NO:20) and GGGGRGDY (SEQ ID NO:21) and FTLCFD (SEQ ID NO:22). In some embodiments, cell attachment peptides comprise RGD, YIGSR (SEQ ID NO:1), IKVAV (SEQ ID NO:2), REDV (SEQ ID NO:3), DGEA (SEQ ID NO:4), VGVAPG (SEQ ID NO:5), GRGDS (SEQ ID NO:6), LDV, RGDV (SEQ ID NO:7), PDSGR (SEQ ID NO:8), RYVVLPR (SEQ ID NO:9), LGTIPG (SEQ ID NO:10), LAG, RGDS (SEQ ID NO:11), RGDF (SEQ ID NO:12), HHLGGALQAGDV (SEQ ID NO:13), VTCG (SEQ ID NO:14), SDGD (SEQ ID NO:15), GREDVY (SEQ ID NO:16), GRGDY (SEQ ID NO:17), GRGDSP (SEQ ID NO:18), VAPG (SEQ ID NO:19), GGGGRGDSP (SEQ ID NO:20) and GGGGRGDY (SEQ ID NO:21) and FTLCFD (SEQ ID NO:22) and further comprise additional amino acids, such as for example, 1-10 additional amino acids, including but not limited 1-10 G residues at the N or C terminal For example, a suitable peptide may have the formula (Xaa)ₙ-SEQ-(Xaa)ₙ wherein Xaa are each independently any amino acid, n=0-7 and SEQ=a peptide sequence selected from the group consisting of: RGD, YIGSR (SEQ ID NO:1), IKVAV (SEQ ID NO:2), REDV (SEQ ID NO:3), DGEA (SEQ ID NO:4), VGVAPG (SEQ ID NO:5), GRGDS (SEQ ID NO:6), LDV, RGDV (SEQ ID NO:7), PDSGR (SEQ ID NO:8), RYVVLPR (SEQ ID NO:9), LGTIPG (SEQ ID NO:10), LAG, RGDS (SEQ ID NO:11), RGDF (SEQ ID NO:12), HHLGGALQAGDV (SEQ ID NO:13), VTCG (SEQ ID NO:14), SDGD (SEQ ID NO:15), GREDVY (SEQ ID NO:16), GRGDY (SEQ ID NO:17), GRGDSP (SEQ ID NO:18), VAPG (SEQ ID NO:19), GGGGRGDSP (SEQ ID NO:20) and GGGGRGDY (SEQ ID NO:21) and FTLCFD (SEQ ID NO:22, and the total number of amino acids is less than 22, preferably less that 20, preferably less that 18, preferably less that 16, preferably less that 14, preferably less that 12, preferably less that 10. Cell attachment peptides comprising the RGD motif may be in some embodiments, 3, 4, 5, 6, 7, 8, 9 or 10 amino acids in length. Examples include, but are not limited to, RGD, GRGDS (SEQ ID NO:6), RGDV (SEQ ID NO:7), RGDS (SEQ ID NO:11), RGDF (SEQ ID NO:12), GRGDY (SEQ ID NO:17), GRGDSP (SEQ ID NO:18), GGGGRGDSP (SEQ ID NO:20) and GGGGRGDY (SEQ ID NO:21). In some embodiments, cell attachment peptides consist of RGD, YIGSR (SEQ ID NO:1), IKVAV (SEQ ID NO:2), REDV (SEQ ID NO:3), DGEA (SEQ ID NO:4), VGVAPG (SEQ ID NO:5), GRGDS (SEQ ID NO:6), LDV, RGDV (SEQ ID NO:7), PDSGR (SEQ ID NO:8), RYVVLPR (SEQ ID NO:9), LGTIPG (SEQ ID NO:10), LAG, RGDS (SEQ ID NO:11), RGDF (SEQ ID NO:12), HHLGGALQAGDV (SEQ ID NO:13), VTCG (SEQ ID NO:14), SDGD (SEQ ID NO:15), GREDVY (SEQ ID NO:16), GRGDY (SEQ ID NO:17), GRGDSP (SEQ ID NO:18), VAPG (SEQ ID NO:19), GGGGRGDSP (SEQ ID NO:20) and GGGGRGDY (SEQ ID NO:21) and FTLCFD (SEQ ID NO:22). In some embodiments in which the cell attachment peptide consists of GRGDY (SEQ ID NO:17), biostructures include less than 2×10⁶ cells/mL or greater than 2×10⁷ cells/mL when produced. In some embodiments in which the cell attachment peptide consists of GRGDY (SEQ ID NO:17), biostructures includes between 2×10⁶ cells/mL and 2×10⁷ cells/mL when produced provided that, in addition to modified alginate comprising an alginate chain section having a cell attachment peptide consisting of GRGDY (SEQ ID NO:17), the modified alginate also comprises the same and/or a different alginate chain section having a cell attachment peptide other than GRGDY (SEQ ID NO:17.

U.S. Pat. No. 6,642,363, which is incorporated herein by reference, discloses covalently linking cell attachment peptides to alginate polymers.

In some embodiments, the purified alginate which comprises covalently linked cell attachment peptides is purified to remove endotoxin. In some embodiments, the purified alginate which comprises covalently linked cell attachment peptides comprises <500 EU/g endotoxin. In some embodiments, the purified alginate which comprises covalently linked cell attachment peptides comprises <250 EU/g endotoxin. In some embodiments, the purified alginate which comprises covalently linked cell attachment peptides comprises <200 EU/g endotoxin. In some embodiments, the purified alginate which comprises covalently linked cell attachment peptides comprises <100 EU/g endotoxin. In some embodiments, the purified alginate which comprises covalently linked cell attachment peptides comprises <50 EU/g endotoxin. In some embodiments in which the cell attachment peptide consists of GRGDY (SEQ ID NO:17), the purified alginate which comprises covalently linked cell attachment peptides comprises <50 EU/g endotoxin. In some embodiments in which the cell attachment peptide consists of GRGDY (SEQ ID NO:17), the purified alginate which comprises covalently linked cell attachment peptides comprises <50 EU/g endotoxin provided that, in addition to the purified alginate having a cell attachment peptide consisting of GRGDY (SEQ ID NO:17), the purified alginate which also comprises the same and/or a different alginate chain section having a cell attachment peptide other than GRGDY (SEQ ID NO:17).

In some embodiments, cells are encapsulated within alginate matrices. The matrices are generally spheroid. In some embodiments, the matrices are irregular shaped. Generally, the alginate matrix must be large enough to accommodate an effective number of cells while being small enough such that the surface area of the exterior surface of the matrix is large enough relative to the volume within the matrix. As used herein, the size of the alginate matrix is generally presented for those matrices that are essentially spheroid and the size is expressed as the largest cross section measurement. In the case of a spherical matrix, such a cross-sectional measurement would be the diameter. In some embodiments, the alginate matrix is spheroid and its size is between about 20 and about 1000 μm. In some embodiments, the size of the alginate matrix is less than 100 μm, e.g. between 20 to 100 μm; in some embodiments, the size of the alginate matrix is greater than 800 μm, e.g. between 800-1000 μm. In some embodiments, the size of the alginate matrix is about 100 μm, in some embodiments, the size of the alginate matrix is about 200 μm, in some embodiments, the size of the alginate matrix is about 300 μm; in some embodiments, the size of the alginate matrix is about 400 μm, in some embodiments, the size of the alginate matrix is about 500 μm; in some embodiments, the size of the alginate matrix is about 600 μm; and in some embodiments about 700 μm.

In some embodiments, the alginate matrix comprises a gelling ion selected from the group Calcium, Barium, Zinc and Copper and combinations thereof. In some embodiments, the alginate polymers of the alginate matrix contain more than 50% α-L-guluronic acid. In some embodiments, the alginate polymers of the alginate matrix contain more than 60% α-L-guluronic acid. In some embodiments, the alginate polymers of the alginate matrix contain 60% to 80% α-L-guluronic acid. In some embodiments, the alginate polymers of the alginate matrix contain 65% to 75% α-L-guluronic acid. In some embodiments, the alginate polymers of the alginate matrix contain more than 70% α-L-guluronic acid. In some embodiments, the alginate polymers of the alginate matrix have an average molecule weight of from 20 to 500 kD. In some embodiments, the alginate polymers of the alginate matrix have an average molecule weight of from 50 to 500 kD. In some embodiments, the alginate polymers of the alginate matrix have an average molecule weight of from 100 to 500 kD.

Cells may be encapsulated over a wide range of concentrations. In some embodiments, cells are entrapped at a concentration of between less than 1×10⁴ cells/ml of alginate to greater than 1×10⁸ cells/ml of alginate. In some embodiments, cells are entrapped at a concentration of between 1×10⁴ cells/ml of alginate and 1×10⁸ cells/ml of alginate. In some embodiments, cells are entrapped at a concentration of between 1×10⁵ cells/ml of alginate and 5×10⁷ cells/ml of alginate. In some embodiments, cells are entrapped at a concentration of between 1×10⁶ cells/ml of alginate and 5×10⁷ cells/ml of alginate. In some embodiments, cells are entrapped at a concentration of between 5×10⁵ cells/ml of alginate and 5×10⁷ cells/ml of alginate. In some embodiments, cells are entrapped at a concentration of between 2×10⁶ cells/ml of alginate and 2×10⁷ cells/ml of alginate. In some embodiments, cells are entrapped at a concentration of between 5×10⁵ cells/ml of alginate and 1×10⁷ cells/ml of alginate. In some embodiments, cells are entrapped at a concentration of between 5×10⁵ cells/ml of alginate and 5×10⁶ cells/ml of alginate. In some embodiments, cells are entrapped at a concentration of about 2×10⁶ cells/ml.

Isolated stem cells may be cultured in alginate-peptide matrices under conditions which support cell proliferation. Using the alginate-peptide matrices as a multi-dimensional substrate, cell populations may be expanded efficiently with a high degree of cell viability.

Populations of stems cells may be subsequently used in the treatment of neurological disorders, such as for example Parkinson disease, HD (Huntington's disease), stroke, mucopolysaccharidosis and MS (Multiple Sclerosis) and in the treatment of injuries involving nerve damage such as spinal injuries. Such stem cells may be isolated from the alginate matrix and implanted into the patient or the stem cells within the matrices may be implanted. Implantation may be made at an appropriate site where they can impart a therapeutic effect as in the brain or spinal column or other site of nerve damage.

In some embodiments, stem cell populations have gene expression characteristics as shown in Table 1. In some embodiments, stem cell populations have gene expression characteristics as shown in Table 2. In some embodiments, stem cell populations have gene expression characteristics as shown in Table 3. In some embodiments, stem cell populations have gene expression characteristics as shown in Supplemental Table 1. In some embodiments, stem cell populations have gene expression characteristics as shown in Supplemental Table 2. In some embodiments, stem cell populations have gene expression characteristics as shown in Supplemental Table 3. In some embodiments, stem cell populations have gene expression characteristics as shown in Supplemental Table 4.

### EXAMPLES

### Example 1

### Entrapment of Human Mesenchymal Stem Cells in Alginate Beads with RGD Peptides

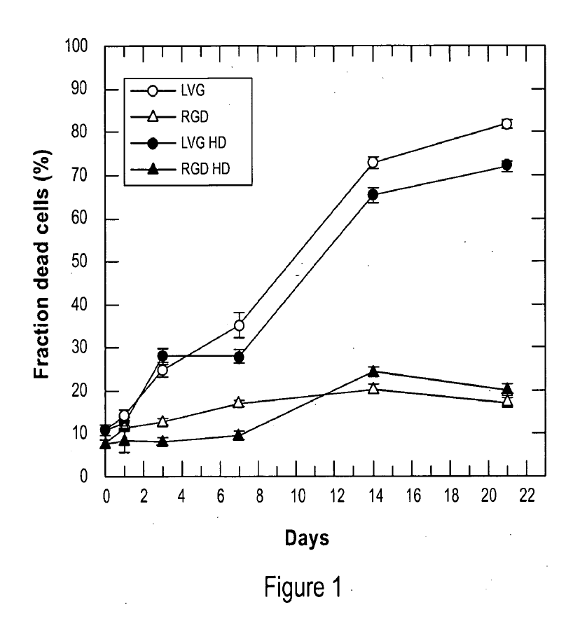

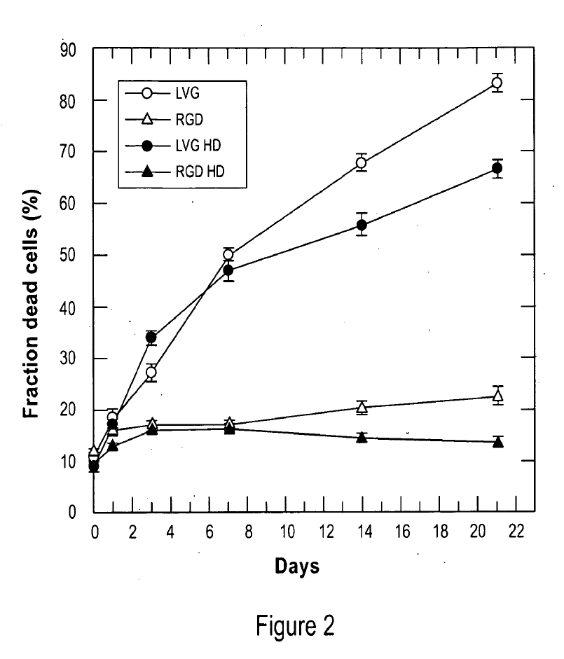

Human mesenchymal stem cells from fat (FIG. 1) and bone marrow (FIG. 2) were isolated from human donors and entrapped in alginate beads. The cells were mixed in solutions of 2% alginate with a high G content (˜70%, PRONOVA LVG) and beads around 400 μm were generated by using a Nisco VAR V1 electrostatic bead generator with a solution of 50 mM CaCl₂ as gelling bath. One of the alginate batches contained RGD peptides covalently linked to the polymer. The cell density was adjusted to be around 80-100 cells/bead in one experiment, and 10-fold higher in another. After gelling, the beads containing the stem cells were stored in tissue culture flasks with cell culture medium in a CO₂ incubator. The fraction of viable and dead cells was at different times calculated by counting cells in a few beads stained with a live dead assay (Molecular Probes, L3224) by using a fluorescence microscope. For both stem cell types it was observed that the total number of cells changed very little throughout the experiment (21 days). However, for both cell types (FIGS. 1 and 2) the number of surviving cells decreased very rapidly for cells entrapped in non RGD-alginate beads. The data thus surprisingly demonstrates that the RGD-alginate cell binding, in addition to the support for cell attachment, is critical in preventing cell death within the alginate gel matrix. The effect of cell to cell interaction on cell survival was also studied in the experiments by increasing the cell concentration 10 fold. As can be seen from the data in FIGS. 1 and 2 there is only a very small or no effect on cell death with time in the LVG alginate beads when increasing the cell concentration. For both cell types the alginate bead cellular density did not have any significant effect on the ability to prevent cell death by the RGD-alginate.

To the extent that the RGD-alginate matrix may improve cell survival, such a property may be an additional property that makes it useful in new biomedical applications with alginate, in particular within tissue engineering, for cell encapsulation and for cultivation of stem cells.

### Example 2

### Demonstration of Inhibited Apoptosis for Bone Marrow Derived Stem Cells Entrapped in RGD-Alginate

Human mesenchymal stem cells from bone marrow were isolated from human donors and grown as a monolayer culture or entrapped in alginate beads using LVG-alginate or RGD-alginate. Entrapment of cells in the alginate was performed as described in Example 1. The alginate cell populations were prepared as single cell suspensions by degelling. BrdU (to a final concentration of 10 μM) is added to the cell culture 1½ h before harvesting by centrifugation at 300×g for 10 minutes at 4° C. The pellet is resuspended in 100 μl ice-cold PBS, and the cells are fixed by adding 70% ethanol (4 ml). The tubes are inverted several times and then stored overnight (at least 18 hours) at −20° C. The cells are then collected by centrifugation, and the pellet is resuspended in pepsin-HCl solution (1 ml). After exactly 30 minutes incubation, the acid is neutralized by adding 0.1 M sodium tetraborate, pH 8.5 (3 ml). The cells are pelleted, washed once with IFA (2-3 ml) and then incubated with IFA-T (2-3 ml) for 5 minutes at room temperature. The cells are again pelleted, resuspended in BrdU-antibody solution (100 n1) and then incubated for at least 30 minutes in a dark place. IFA-T (2-3 ml) is added to the cell suspension, and the cells are then pelleted before they are resuspended in RNase/PI solution (500 μl). After 10 minutes incubation, the cell suspension is transferred to a Polystyrene Round-Bottom Tube (5 ml). The cells are analyzed in the flow cytometer.

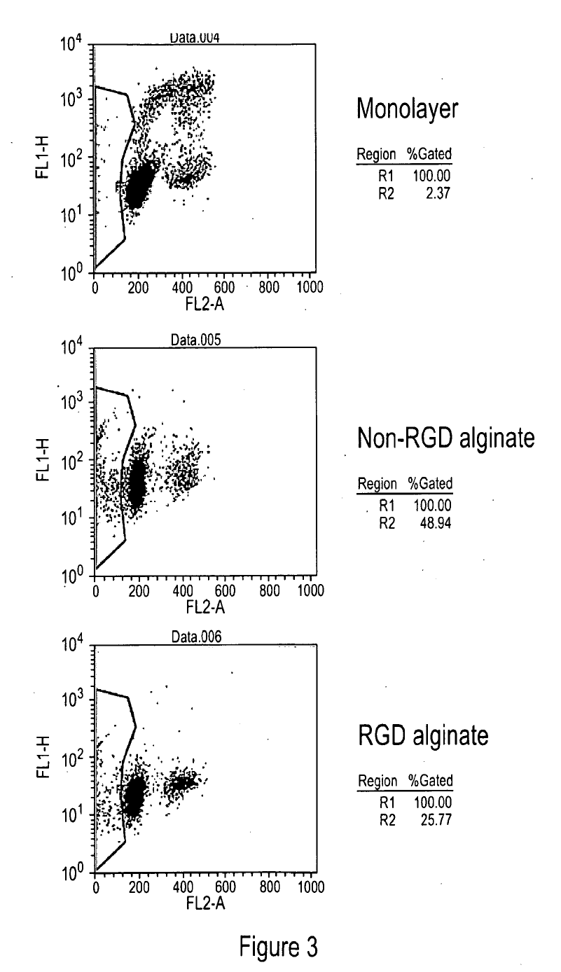

In FIG. 3 two parametric recordings are shown for cells after 6 days. In contrast to cells grown as monolayers the number of actively proliferating cells (BrdU positive cells) is shown to be very low for the alginate entrapped cell cultures. Also for these cells there was an increased fraction of dead cells with a sub G1 DNA content (R2-gates in FIG. 3) indicating apoptotic activity in the alginate populations. The fraction of sub G1 cells was, however, reduced by approximately 50% in the RGD alginate as compared to non RGD-alginate sample (FIG. 3). The data thus clearly indicated that DNA degradation was more inhibited for cells grown in the RGD alginate environment versus non-RGD alginate. The observation that apoptotic cell death seemed to be inhibited by using RGD in the alginate matrix was also further supported by independent data using a TUNEL assay. Our experiments thus clearly indicated that cell attachment, as supported by the RGD bound alginate, prevented apoptotic activity in the stem cell populations.

### Example 3

### Materials and Methods

### Isolation of AT-MSC

AT was obtained by liposuction from healthy donors aged 18-39. The donors provided written informed consent, and the collection and storage of adipose tissue (AT) and AT-MSC was approved by the regional committee for ethics in medical research in Norway. The stromal vascular fraction (SVF) was separated from AT as described previously {Boquest, 2005 2900/id}. Briefly, lipoaspirate (300-1000 ml) was washed repeatedly with Hanks' balanced salt solution (HBSS) without phenol red (Life Technologies-BRL, Paisley, UK) containing 100 IU/ml penicillin and 100 IU/ml streptomycin (Sigma Aldrich, St. Louis, USA) and 2.5 mg/ml amphotericin B (Sigma). Washed AT was digested for 45 min on a shaker at 37° C. using 0.1% collagenase A type 1 (Sigma) After centrifugation at 400 g for 10 min, floating adipocytes were removed. The remaining SVF cells were resuspended in HBSS containing 2% fetal bovine serum (FBS). Tissue clumps were allowed to settle for 1 min. Suspended cells were filtered through 100 μm and then 40 μm cell sieves (Becton Dickinson, San Jose, Calif.). Cell suspensions (15 ml) were layered onto 15 ml Lymphoprep gradient separation medium (Axis Shield, Oslo, Norway) in 50-ml tubes. After centrifugation (400 g, 30 min), cells at the gradient interface were collected, washed and resuspended in regular medium containing 10% FBS and antibiotics. Cell counts and viability assessment were performed using acridine orange/ethidium bromide staining and a fluorescence microscope.

Immediately after separation, AT-MSC were isolated from the remaining cells using magnetic cell sorting. Endothelial cells (CD31⁺) and leukocytes (CD45⁺) were removed using magnetic beads directly coupled to mouse anti-human CD31 and CD45 monoclonal antibodies (MAb) (Miltenyi Biotech, Bergish Gladbach, Germany) and LS columns. For verification, we measured by flow cytometry and observed that no more than 5% of CD31+ and CD45+ cells were left in the suspension. Cells were washed and resuspended in Dulbecco's modified Eagle's medium (DMEM)/F12 (Gibco, Paisley, U.K.) containing 20% FBS and antibiotics.

### Isolation of BM-MSC

Bone Marrow (BM) (100 ml) was obtained from the iliac crest of healthy voluntary donors after written informed consent. The collection and storage of BM and BM-MSC was approved by the regional committee for ethics in medical research. The aspirate was diluted 1:3 with medium. Cell suspensions (15 ml) were applied to 15 ml Lymphoprep gradients in 50-ml tubes. After density-gradient centrifugation at 800 g for 20 minutes, the mononuclear cell layer was removed from the interface, washed twice, and suspended in DMEM/F12 at 10⁷ cells per ml. To reduce the occurrence of other adherent cells, monocytes were removed using magnetic beads coupled to mouse anti-human CD14 MAb according to the manufacturer's recommendations (Miltenyi). The CD14⁻ cells were washed and allowed to adhere overnight at 37° C. with 5% humidified CO₂ in culture flasks (Nunc, Roskilde, Denmark) in DMEM/F12 medium with 20% EBS and antibiotics.

### Culturing of BM-MSC and A T-MSC

On day 1 of BM-MSC cultures the medium with nonadherent cells was discarded, the cultures were carefully washed in DPBS (Gibco), and culture medium was replaced with a fresh portion. When the cells reached 50% confluence, plastic adherence was interrupted with trypsin-EDTA (Sigma), and the cells were inoculated into new flasks at 5,000 cells per cm². After the first passage, amphotericin B was removed and 10% FBS was used in stead of 20% for the duration of the cultures. Viable cells were counted at each passage. The medium was replaced every 2-3 days.

### Preparation and Use of Alginate Gels

Low viscosity, high guluronic acid sodium alginate (Pronova LVG, MW 134 kDa, here termed regular alginate), and custom made GRGDSP alginate (Novatech RGD, peptide/alginate molecular ratio of approximately 10/1) made from high guluronic acid alginate (Pronova UP MVG, MW 291 kDa) was obtained from NovaMatrix/FMC Biopolymer (Oslo, Norway). The guluronic-mannuronic acid ratio in all cases was ˜70:30 ratio. A 2% alginate solution was prepared by dissolving the alginate powder in a 250 mM mannitol solution and was stirred overnight at room temperature before the solution was filtered through a 0.22 μM filter.

Prior to encapsulation in alginate, monolayer AT-MSC and BM-MSC at 50% confluence were trypsinized and suspended in 500 μl medium. The cells were mixed into the appropriate alginate solution at 0.5, 2.0 or 5.0×10⁶ cells/ml. The cell/alginate suspension was gelled as beads using an electrostatic bead generator (disco VAR V1, Zurich, Switzerland). Beads were generated at 6 kV/cm and 10 ml/hr using a 0.5 mm (outer diameter) nozzle, and crosslinked in a 50 mM CaCl₂ solution. After storing the beads in the gelling solution for approx 20 minutes they were washed with medium several times and kept in culture flasks using DMEM/F12 medium containing 10% FBS and antibiotics. The beads with MSC were maintained in culture for 21 days and medium was changed every third day. The beads were soaked in sterile-filtered 50 mM CaCl₂ every seventh day. For being able to perform different analyses different time points the cells were released from the alginate beads by washing with a 100 mM EDTA-DPBS solution for five minutes and centrifuged at 1500 rpm for 15 min. Finally the cells were resuspended in DPBS (Gibco) and analyzed in different assays.

### Viability Assay

Live/Dead viability assay (Invitrogen Molecular Probes, Eugene, Oreg., USA) was performed on the alginated cells. Briefly, beads were allowed to settle and were washed with DPBS. Cells were incubated with 8 μl of Component B (2 mM Ethidium bromide stock solution) and 2 μl of Component A (4 mM of Calcein AM stock solution) in 2 ml of 4.6% sterile no mannitol solution, at room temperature for 45 min in the dark. Cells were examined and counted under a fluorescence microscope, altering the focal distance to allow assessment of all the cells in the beads. For each assay 15-20 beads were included in the evaluation. This assay was performed on day 0, 1, 3, 7, 14 and 21 following encapsulation in alginate.

### Apoptosis Assay

TUNEL assay to check for apoptosis was performed on cells that had been cultured in unmodified and RGD alginate for 7 days using an In Situ Cell Death Detection Kit (Roche Diagnostics Ltd, Burgess Hill, UK). Briefly, the alginate beads were degelled as described above, leaving the cells in single cell suspension. The cells were fixed with 4% (w/v) paraformaldehyde and incubated on ice for 15 min. Fixed cells were washed with DPBS, resuspended in 200 μl of 0.1% saponin and incubated for 15 minutes to permeabilise the cells (ice). After washing, the resuspended cells were incubated with 50 μl TUNEL reaction mixture for 1 hour at 37° C. in the dark (ice). The cells were then washed, resuspended in 200 μl of PBS and examined in a fluorescence microscope.

### BrdU Assay

The incorporation of BrdU in monolayer cells and cells in beads were analyzed at day 7. 3×10⁵ cells in monolayer and within alginate beads, respectively, were pulsed with 10 μM of BrdU for two hrs. Then monolayer cells were trypsinized, while encapsulated cells were degelled with CaCl₂ and washed with DPBS. The cells were fixed in 70% ethanol and stored at −20° C. After 24 hrs cells were collected by centrifugation at 400 g for 5 min, and then resuspended in pepsin-HCl solution for 1 hr followed by neutralization by 0.1 M sodium tetraborate, pH 8.5 (3 ml). The cells were washed once with immunofluorescence assay buffer (IFA) (2-3 ml) and then incubated with IFA containing Tween 20 (2-3 ml) for 5 minutes at room temperature before staining with a FITC-conjugated anti-BrdU MAb (BD Biosciences) and propidium iodide. Cells were analyzed using a FACSCalibur flowcytometer (BD Biosciences).

### Isolation of Resting CD8+ T Cells

Resting CD8+ T cells were used as control population which does not proliferate in ³H thymidine incorporation assays. The cells were isolated from peripheral blood mononuclear cells using negative isolation with a Pan T Isolation Kit, CD4 MACS beads, LS columns and a SuperMACS magnet as described by the producer (Miltenyi Biotech)

### Thymidine Incorporation Assay

The uptake of ³H thymidine, a measure of DNA synthesis, was examined on day 7 in 5 different donors for AT-MSC and 3 donors for BM-MSC. Trypsinized monolayer cells and MSC in beads were seeded at 15.000 cells per well in 96 flat bottom well plates, pulsed with 1 μCi ³H thymidine in 200 μl of DMEM/F12 medium containing 10% FBS and antibiotics in each well and incubated at 37° C. in 5% CO₂ for 24 hrs. The amount of ³H thymidine that had been incorporated into the DNA cells was measured using a TopCount NXT Scintillation counter (Packard, Meriden, Conn.).

### Cell Surface Markers Analysis

Monolayer and degelled MSC from beads were analyzed at day 7 for cell surface markers by flowcytometry. Cells were stained with unconjugated MAbs directed against the following proteins: CD49e, CD 29, CD49c, CD61, CD51, CD41 (kind gift from Dr. F. L. Johansen). For immunolabeling, cells were incubated with primary MAbs for 15 min on ice, washed, and incubated with PE-conjugated goat anti-mouse antibodies (Southern Biotechnology Association, Birmingham, Ala.) for 15 min on ice. After washing, cells were analyzed by flowcytometry (FACSCalibur)

### Results

### MSC Die in Cultures of Regular Alginate

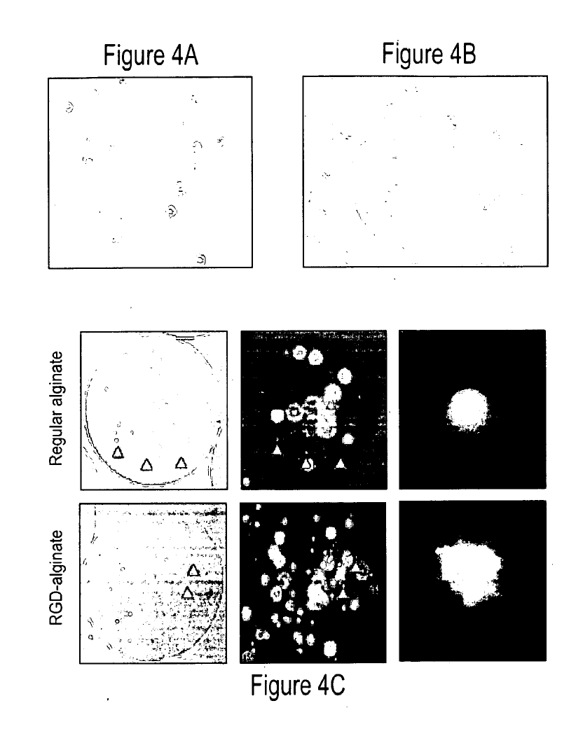

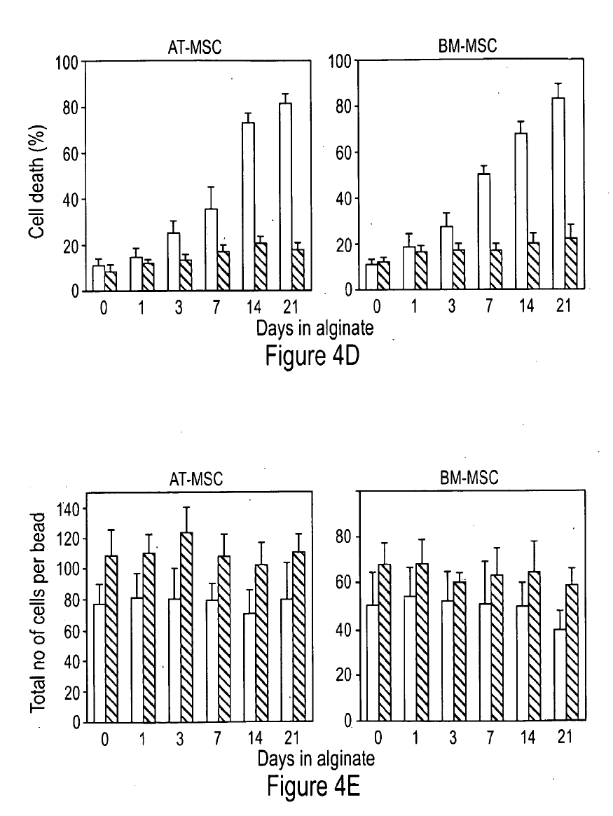

Immediately Upon Isolation From Adipose Tissue, At-Msc Have a Small, regular, rounded shape (FIG. 4A). Following attachment, spreading and proliferation on plastic surfaces, they acquired a long, spindle-like shape (FIG. 4B). To determine if, when the attachment to the underlying plastic surface was disrupted, the cells would get their previous shape, cells were entrapped in alginate, which consists of long chains of α-L-guluronic acid and β-D-mannuronic acid, and which provides an inert scaffold around the cells. The result is visualized in FIG. 4C, upper panel. MSC cultured in this 3D system were found to be small and round. We also observed that MSC cultured in regular alginate showed a high proportion of dead cells after some time in culture. Those were seen as red cells in the LIVE/DEAD assay (FIG. 4C, upper middle image). The proportion of live and dead cells in cultures in regular alginate was quantified and is shown in FIG. 4D, grey bars. After three weeks in culture, the vast majority of cells had died. These cells remained in the alginate as countable cells, since the variation of total number of cells was negligible in the course of these three weeks of culture (FIG. 4E, grey bars). Similar results were obtained for BM-MSC. We thought it might be possible that the cell density in the alginate might influence the live/dead outcome, so we performed the same experiment, and compared number of dead cells in beads made of 0.5×10⁶ cells/ml of alginate (used in the previous experiments) with number of dead cells in beads made of 5×10⁶ cells/ml of alginate. However, the results were essentially the same, both for AT-MSC and BM-MSC (data not shown). For the rest of these experiments, we chose to encapsulate MSC in alginate at the concentration of 2×10⁶ cells/ml.

### RGD Binding to Integrin Molecules on MSC Ensures Cell Survival/Inhibits Cell Death in Alginate Cultures

The tripeptide RGD is found in several of the molecules in the ECM, binds to integrin heterodimers on the cell surface and is important for cell survival through which intracellular signals {Frisch, 1997 3134/id}. We embedded MSC in alginate into which the RGD peptide had been incorporated. Here, the cells still had a small and fairly rounded shape, but extensions from the body of the cells could frequently be observed, suggesting attachment to the surrounding material (FIG. 4C, lower right panel). Dead (red) cells could still be observed in the live/dead assay, but not nearly as many as with regular alginate (FIG. 4C, lower middle panel). Quantification of live and dead cells in the RGD alginate cultures is shown in FIG. 4D, black bars, and shows that 10-15% of the cells died in encapsulation. There was no evidence of an increase in the total number of cells over this culture period (FIG. 4E). Similar results were obtained for AT-MSC and BM-MSC.

### MSC in Regular Alginate Most Likely Die by Programmed Cell Death

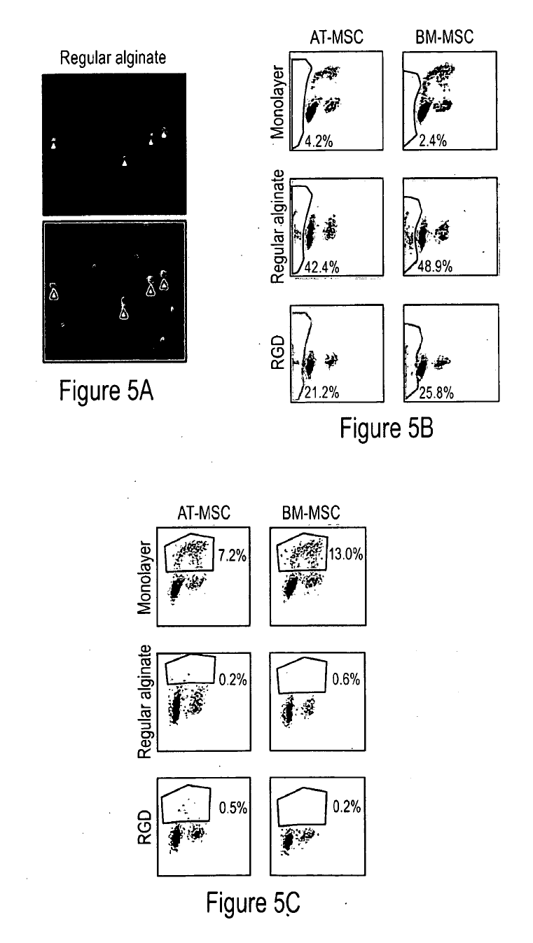

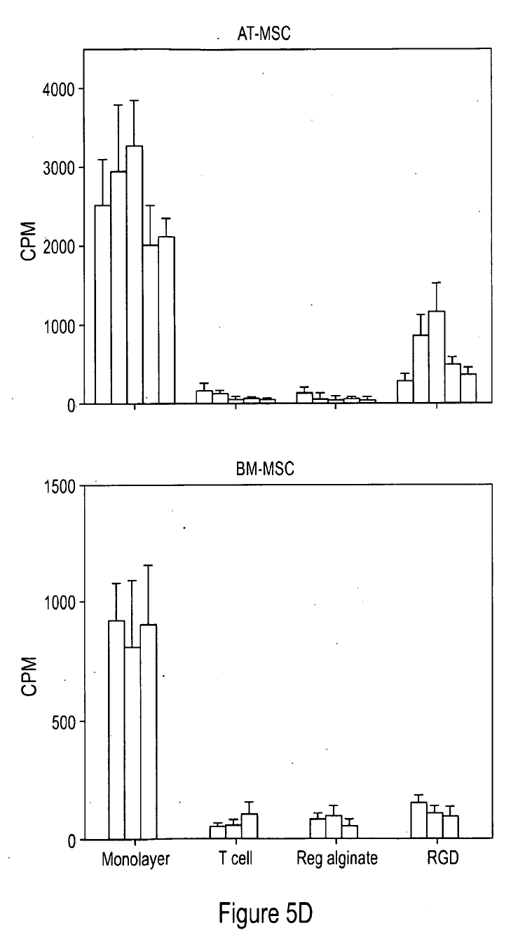

In order to determine type of cell death was initiated in regular alginate, we performed TUNEL assay at day 7. Results for AT-MSC are shown in FIG. 5A. The proportion of TUNEL+ cells in this assay identifies cells with endonuclease-mediated DNA strand breaks (double-stranded), and indicates that these cells die by programmed cell death (PCD). Similar results were observed with BM-MSC (data not shown).

The presence of short DNA strands, indicative of DNA fragmentation into oligonucleosomal subunits, can be visualized and quantified as a subG1 population by flow cytometry. BrdU staining of MSC on day 7, cultured in 2D and 3D, gated for subG1 populations, is shown in FIG. 5B. Only 2-4% of the cells cultured in monolayer were found in the subG1 population, indicating a small proportion of cell death. Of the cells in regular alginate, 42 and 49% were found in the subG1 population for AT- and BM-MSC respectively, while 21 and 26% of the cells in RGD alginate were in the sub G1 population for AT- and BM-MSC respectively. This further indicates PCD as the mode of death, and substantiates the results from the LIVE/DEAD assay.

### Modest Proliferation of AT-MSC and No Proliferation of BM-MSC in 3D Alginate Cultures

Results from cell counts suggested that MSC embedded in alginate did not proliferate. We used the BrdU assay to estimate numbers of cells that were in S-phase, which would reflect the level of proliferation. A high proportion of the cells cultured in monolayer was found to be in the S phase of cell cycle, while the proportion of encapsulated cells in S phase was very low, similar to that previously described for uncultured AT-MSC {Boquest, 2006 3128/id} Another way to estimate proliferation is by measuring ³H thymidine incorporation. FIG. 5D shows this assay performed on cells from 5 donors for AT-MSC and 3 donors for BM-MSC on day 7-8 of culture. There was high uptake of ³H thymidine in all the cells cultured in monolayer, confirming high proliferative activity. No activity was observed for the MSC cultured in regular alginate. However, for AT-MSC cultured in RGD alginate we observed a small/moderate uptake of 3H thymidine.

### MSC Cultured in RGD Alginate Retain Expression of Integrins Involved in Binding to RGD-Containing ECM Proteins

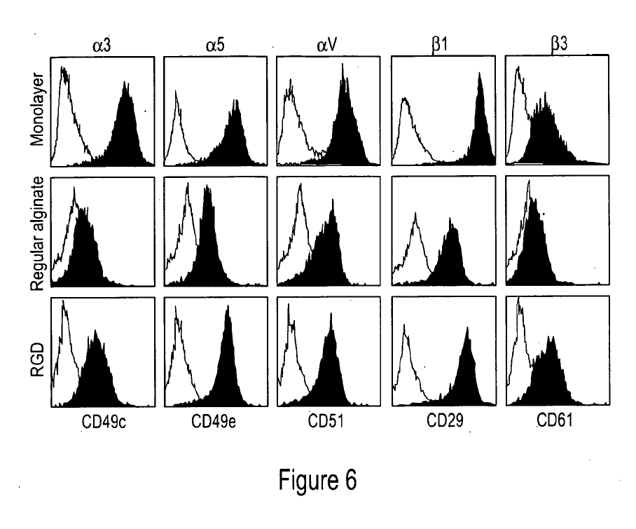

A number of integrin heterodimers are known to be involved in binding to the RGD motif in ECM molecules. To determine if embedding of MSC in alginate affected the expression of integrins on the cell surface, we used flow cytometry to detect the expression level of some of the integrin monomers involved in RGD binding. The results are shown in FIG. 6. MSC cultured in monolayer showed high expression of these molecules, suggesting that perhaps these molecules are of importance for their attachment to plastic. Following 7 days of culture in regular alginate, all these integrins were down-regulated. All the integrins, except CD61, were also down regulated in MSC cultured in RGD alginate, but to a lesser extent than on the cells cultured in regular alginate.

### Example 4

### Entrapment of MSC in RGD Alginate Induces Changes in Gene Expression

Human mesenchymal stem cells from bone marrow and adipose tissue (AT) were isolated from human donors and grown as a monolayer culture and later entrapped in alginate beads using LVG-alginate or RGD-alginate. Entrapment of cells in the alginate was performed as described in Example 1. At different times the cells were released from the alginate beads by washing with DPBS (Gibco) containing 100 mM EDTA for five minutes and centrifuged at 1500 rpm for 15 min. Finally the cells were resuspended in DPBS (Gibco) and further analyzed.

RNA sample preparation and microarray assay were performed according to the Affymetrix GeneChip Expression Analysis Technical Manual (Affymetrix, Santa Clara, Calif.). Briefly, freshly isolated AT-MSC, monolayer cultured and degelled alginate encapsulated cells from three donors at day 7 each were pelleted and snap frozen in liquid nitrogen. Total RNA was extracted from cells using Ambion RNaqueous (Miro, Austin, Tex.). Due to small amounts of RNA in freshly isolated uncultured cells, cDNA was prepared from 100 ng of total RNA using the Two-Cycle cDNA Synthesis Kit (Affymetrix P/N 900432). For all samples, 10 μg of cRNA 10 was hybridized to the HG-U133A_{—}2 array (Affymetrix) representing 22,277 probes. Arrays were scanned with Affymetrix GeneChip Scanner 3000 7G. The data are published in ArrayExpress, accession number E-MEXP-1273.

The open-source programming language and environment R (http://crans-project.org/doc/FAQ/RFAQ.html#Citing-R) was used for pre-processing and statistical analysis of the Affymetrix GeneChip microarrays. The Bioconductor {Gentleman, 2004 3127/id} community builds and maintains numerous packages for microarray analysis written in R, and several were used in this analysis. First, the array data were normalized using the gcRMA package {Wu Z, 2004 3129/id}. Then probes with absent calls in all arrays were discarded from the analysis. After preprocessing and normalization, a linear model of the experiment was made using Limma. This program was also used for statistical testing and ranking of significantly differentially expressed probes {Smyth G K, 2004 3130/id}. Affy was used for diagnostic plots and filtering {Gautier L, 2004 3131/id}. To adjust for multiple testing, the results for individual probes were ranked by Benjamini-Hochberg {Benjamini, 1995 3132/id} adjusted p-values, where p<0.01 was considered significant.

As changes in cell shape, polarity and proliferation has been shown to strongly influence gene expression {Yamada, 2007 3126/id}, we wanted to determine the changes in global mRNA expression observed between cells where all these factors were changed. To our surprise, we found no significant difference at the mRNA expression level between cells entrapped in RGD and regular alginate using Benjamini Hochberg multiple testing with p<0.01 (data not shown). This suggests that the events involved in PCD in these cells all occur at the post-transcriptional level.

For our analysis of differentially expressed genes, using p<0.01 and >3-fold change, we found probes representing 48 genes to be up-regulated upon entrapment in alginate. Gene ontology analysis showed that these genes could be functionally associated with cell adhesion and a number of metabolic processes (Supplementary Table 1). The list of upregulated genes is given in Table 1. The most highly upregulated gene, CNIH, encodes a protein associated with polarization of the cytoskeleton {Roth, 1995 3120/id}. Other genes associated with the cytoskeleton and actin-myosin association are MLPH, ARL4C, and FHOD3. An integrin (133,CD61) was found to be moderately upregulated at the mRNA level. The expression of R3 at the protein level was also slightly increased in MSC in RGD alginate compared with cells cultured in monolayer, consistent with the observed up-regulation at the mRNA level. Interestingly, the TDO2 gene was greatly upregulated in RGD alginate entrapped cells. The gene product, tryptophan 2,3-dioxygenase, is involved in the catabolism of tryptophan {Takikawa, 2005 3118/id}. The accelerated breakdown of tryptophan has been suggested to be an important mechanism for the immunosuppressive effect mediated by MSC {Meisel, 2004 2851/id}.

The gene ontology of the 39 genes downregulated in AT-MSC following entrapment in RGDalginate is shown in Supplementary Table 2. The largest clusters of genes were those associated with development, intracellular signaling and cellular morphogenesis. The list of individual genes is given in Table 2. It contains a number of genes associated with the cytoskeleton and filament biology (KRT18, FLG, CDC42EP3, VIL2, CAP2, FHL1, LMO7 and MFAPS). Three of the genes were associated with the cell cycle (TPD52L1, NEK2 and SEP11), while some genes were associated with lineage differentiation (HAPLN1 for cartilage; MEST and ZFP36 15 for fat; OXTR, ACTC, TRPC4, ACTA2 and PDE1C for cardiovascular and muscle; and RGS7 and MBP for neuronal differentiation).

Supplementary Table 3 shows the gene ontology of 665 probes representing genes upregulated in alginate entrapped cells. The vast majority of the most significantly upregulated probes represent genes associated with a range of metabolic processes. Also highly significant were categories of genes regulating macromolecule biosynthesis and cell localization and adhesion. MMP1 can be found at the top of the list of individual genes overexpressed in alginate entrapped cells (Table 3), but a number of other genes associated with the ECM (COMP, COL11A1, PAPPA, FN1, LTBP1) were also highly upregulated in these cells. Other functionally clustered genes on this shortlist are some involved with the cytoskeleton (LPXN, DSP, MICAL2) and with the bone morphogenic protein (BMP) pathway (GREM2, GREM1, TRIB3, LTBP1). TMEM158 and ITGA10 were found as highly upregulated in alginate entrapped cells both in comparison with cells cultured in monolayer and with uncultured cells, suggesting that these genes are specifically upregulated as a result of entrapment in RGD alginate.

Compared with MSC entrapped in RGD alginate, prospectively isolated, uncultured AT-MSC overexpressed genes clustered as associated with development and differentiation to a number of lineages. Supplementary Table 4 shows the gene ontology of the 503 probes which were upregulated in the uncultured cells. On the list of the most highly upregulated individual genes, CXCL14 ranks highest, followed by the BMP antagonist CHRDL1. Substantiating the gene ontology list, a number of genes associated with fat (CFD, APOD, SEPP1, FABP4, C7, LPL 16 and AADAC) and osteochondral differentiation (SPARCL1, ITM2A, CILP, SERPINA3, OMD and OGN) were found.

To this end, a wide range of 2D and 3D tissue culture procedures have been described. For MSC, practically all published data are based on cells in 2D culture. This is because attachment to a plastic surface is required for the cells to proliferate to yield the cell numbers required for assays or treatment protocols, and also because passage on plastic surfaces selects for the cell population now defined as MSC {Dominici, 2006 3043/id}. However, the change in morphology, polarization of the cytoskeleton, attachment properties and rate of cell division induced by plastic adherence leads to dramatic changes in MSC biology {Yamada, 2007 3126/id} {Boquest, 2005 2900/id}. The hypothesis driving the present invention was that it might be possible to reverse many of these changes by transferring monolayer expanded MSC to 3D cultures. We found that, for MSC in 3D cultures, cell shape, size and rate of cell division were similar to those observed for uncultured MSC {Boquest, 2005 2900/id} {Boquest, 2006 3128/id}. However, under the conditions provided in the present work, the transcriptome of the MSC expanded in 2D and then established in 3D culture was still far removed from that observed in freshly isolated, uncultured AT-MSC. While they could be seen to be closer to the plastic-adherent cells than to the freshly isolated MSC, the gene expression profile of the MSC in 3D cultures suggests that they should be considered to be a separate, third population of MSC.

### Example 5

### Prophetic Example. Using Autologous Stem Cells Entrapped in Alginate in the Treatment in Multiple Sclerosis (MS)

The previous examples describes that MSCs can be expanded to high numbers on plastic surfaces (2D), and then entrapped in alginate and if the tripeptide RGD is incorporated in the alginate, the cells survive over the duration of the study with high viability. The global gene expression analyses (Example 4) demonstrates that the alginate entrapped cells are different from the cells cultured in 2D, and different from cells characterized immediately after isolation, in the uncultured form (Duggal et al., unpublished). These cells seem to represent a new, third population of MSC. For therapeutic purposes, the alginate may be entirely removed, leaving the cells in single cell suspension with the morphological and molecular characteristics of 3D cells.

For cells cultured in alginate to be better than cells cultured in 2D in the treatment of MS, they need to be available at the site of damage in higher numbers, or exert higher efficacy at the site of damage, or be less likely to produce harmful effects, or any combination of these. The strategy for the use of MSC in MS could be based on intravenous (IV) injection or other administration of the cells. MSC cultured in 2D are large cells expressing a high density of adhesion molecules following their adherence to the plastic surface. This is likely to be the main reason why, following IV injection, these cells are retained in the first capillary network that they encounter, which is the pulmonary network. Here, many of the MSC die (see for instance Kraitchmann et al., Circulation 2005; 112:1451). In our work, we have shown that MSC after culture in alginate are smaller, and express a lower concentration of all the integrins tested so far (α3, 5 and V, β1 and 3). Thus, the cells may have a higher chance of escaping through the pulmonary circulation.

The exact mechanism of action of the MSC reported to be efficacious in neurological diseases is not known, but is likely to include immunosuppressive effects, transdifferentiation to neurons, glial cells and oligodendrocytes, and remyelination. For the immunosuppressive effect exerted by MSC, the mechanism of action again is not fully described. However, the induction of an accelerated degradation of tryptophan has been suggested to be of major importance (Meisel et al., Blood 2004; 103:4619). One mechanism by which the alginate entrapped MSC may be superior to the MSC expanded in 2D is through the action of the enzyme tryptophan 2,3-dioxygenase (TDO), which catalyzes the degradation of tryptophan (Murray, Curr Drug Metab 2007; 8:197), and is upregulated approximately 100-fold at the mRNA level in alginate entrapped MSC compared with 2D MSC (Example 4). For the other possible mechanisms of action of MSC no molecular mechanisms are described. Possibly a pre-clinical and clinical trials may show that alginate entrapped MSC have an advantage in these areas. There is precedence for cells cultured in 3D being better than their 2D counterparts for clinical applications. For instance, MSC need to be cultured in 3D to differentiate to chondrocytes (Sekiya et al., PNAS 2002; 99:4397). Another example is the differentiation of myoblasts to muscle tissue (Hill et al., PNAS 2006; 103:2494).

TABLE 1
Genes upregulated in MSC expanded in monolayer and then entrapped
in alginate compared with MSC only expanded in monolayer.
Selection criteria: p < 0.01, >3-fold difference

| | | Fold |
|---|---|---|
| Symbol | Description | change |
| CNIH3 | cornichon homolog 3 | 237 |
| ETV1 | ets variant gene 1 | 112 |
| ITGA10 | integrin, alpha 10 | 88 |
| TDO2 | tryptophan 2,3-dioxygenase | 83 |
| TMEM158 | transmembrane protein 158 | 80 |
| ARHGAP22 | Rho GTPase activating protein 22 | 59 |
| LIPG | lipase, endothelial | 58 |
| SNED1 | sushi, nidogen and EGF-like domains 1 | 43 |
| CLGN | calmegin | 40 |
| DUSP4 | dual specificity phosphatase 4 | 39 |
| MLPH | melanophilin | 33 |
| RNF144 | ring finger protein 144 | 32 |
| GPNMB | glycoprotein nmb | 29 |
| ANGPTL2 | angiopoietin-like 2 | 27 |
| NBL1 | neuroblastoma, suppression of tumorigenicity 1 | 26 |
| ITGA2 | integrin, alpha 2 (CD49B) | 24 |
| PTGER2 | prostaglandin E receptor 2 (subtype EP2) | 23 |
| ENOSF1 | enolase superfamily member 1 | 21 |
| KIAA1644 | KIAA1644 | 20 |
| ARL4C | ADP-ribosylation factor-like 4C | 20 |
| THBD | thrombomodulin | 18 |
| RNF128 | ring finger protein128 | 17 |
| ENO2 | enolase 2 | 17 |
| CTSK | cathepsin K | 15 |
| SLC6A8 | solute carrier family 6 member 8 | 14 |
| PHLDA1 | pleckstrin homology-like domain, family | 13 |
| | A, 1 | |
| COL7A1 | collagen, type VII, alpha 1 | 12 |
| SRPX2 | sushi-repeat-containing protein, X-linked 2 | 11 |
| SLC7A8 | solute carrier family 7, member 8 | 11 |
| FOXO1A | forkhead box O1A | 11 |
| AMY1A | amylase, alpha 1 | 10 |
| SOX4 | SRY (sex determining region Y)-box 4 | 10 |
| ITGB3 | integrin, beta 3 (CD61) | 9 |
| SYNJ2 | synaptojanin 2 | 7 |
| FHOD3 | formin homology 2 domain containing 3 | 7 |
| GPR177 | G protein-coupled receptor 177 | 6 |
| PPFIBP1 | PTPRF interacting protein, binding protein 1 | 6 |
| HS2ST1 | heparan sulfate 2-O-sulfotransferase 1 | 6 |
| C1orf107 | chromosome 1 open reading frame 107 | 6 |
| CYLD | cylindromatosis | 5 |
| ANKRD10 | ankyrin repeat domain 10 | 5 |
| WWOX | WW domain containing oxidoreductase | 5 |
| LPIN1 | lipin 1 | 4 |
| HIC2 | hypermethylated in cancer 2 | 4 |
| SLC2A6 | solute carrier family 2, member 6 | 4 |
| DNMBP | dynamin binding protein | 3 |
| GNPDA1 | glucosamine-6-phosphate deaminase 1 | 3 |
| STAG2 | stromal antigen 2 | 3 |

TABLE 2
Genes downregulated in AT-MSC expanded in monolayer and then
entrapped in alginate compared with AT-MSC only expanded
in monolayer. Selection criteria: p < 0.01, >3-fold difference

| | | Fold |
|---|---|---|
| Symbol | Description | change |
| HAPLN1 | hyaluronan and proteoglycan link | 338 |
| | protein 1 | |
| KRT18 | keratin 18 | 335 |
| MEST | mesoderm specific transcript | 267 |
| | homolog | |
| OXTR | oxytocin receptor | 244 |
| SERPINB7 | serpin peptidase inhibitor, clade B, | 138 |
| | member 7 | |
| ACTC | actin, alpha, cardiac muscle | 93 |
| TRPC4 | transient receptor potential cation channel, | 68 |
| | subfamily C, 4 | |
| B3GALT2 | UDP-Gal:betaGlcNAc beta | 48 |
| | 1,3-galactosyltransferase 2 | |
| RGS7 | regulator of G-protein signalling 7 | 34 |
| MBP | myelin basic protein | 28 |
| SCN9A | sodium channel, voltage-gated, type | 24 |
| | IX, alpha | |
| NPR3 | natriuretic peptide receptor C/guanylate | 23 |
| | cyclase C | |
| FLG | filaggrin | 21 |
| IL7R | interleukin 7 receptor | 20 |
| TPD52L1 | tumor protein D52-like 1 | 19 |
| DKFZP686A01247 | hypothetical protein | 16 |
| ACTA2 | actin, alpha 2, smooth muscle, aorta | 14 |
| C5orf23 | chromosome 5 open reading frame 23 | 12 |
| CDC42EP3 | CDC42 effector protein 3 | 11 |
| PRPS1 | phosphoribosyl pyrophosphate | 11 |
| | synthetase 1 | |
| SH2D4A | SH2 domain containing 4A | 11 |
| PRSS23 | protease, serine, 23 | 10 |
| VIL2 | villin 2 (ezrin) | 10 |
| CAP2 | CAP, adenylate cyclase-associated | 9 |
| | protein, 2 | |
| ZFP36 | zinc finger protein 36 | 8 |
| FHL1 | four and a half LIM domains 1 | 8 |
| ELL2 | elongation factor, RNA polymerase II, 2 | 7 |
| RRAS2 | related RAS viral (r-ras) oncogene | 7 |
| | homolog 2 | |
| RBMS2 | RNA binding moti 2 | 7 |
| LMO7 | LIM domain 7 | 6 |
| DBNDD2 | dysbindin domain containing 2 | 6 |
| NEK7 | NIMA (never in mitosis gene a)-related | 6 |
| | kinase 7 | |
| SEP11 | septin 11 | 5 |
| PDE1C | phosphodiesterase 1C | 5 |
| CHAC1 | ChaC, cation transport regulator-like 1 | 5 |
| TMPO | thymopoietin | 4 |
| IDE | insulin-degrading enzyme | 4 |
| MFAP5 | microfibrillar associated protein 5 | 4 |
| MBNL2 | muscleblind-like 2 | 4 |

TABLE 3
Genes upregulated in AT-MSC expanded in monolayer and then
entrapped in alginate compared with uncultured AT-MSC.
Selection criteria: p < 0.01, top 30 genes by fold change

| | | Fold |
|---|---|---|
| Symbol | Description | change |
| MMP1 | matrix metallopeptidase 1 | 5557 |
| KIAA1199 | KIAA1199 | 1563 |
| INHBA | inhibin, beta A (activin A) | 1243 |
| COMP | cartilage oligomeric matrix protein | 744 |
| HMGA2 | high mobility group AT-hook 2 | 458 |
| LPXN | leupaxin | 393 |
| SLC7A11 | solute carrier family 7, member 11 | 343 |
| DSP | desmoplakin | 290 |
| IL1RN | interleukin 1 receptor antagonist | 288 |
| STC1 | stanniocalcin 1 | 252 |
| COL11A1 | collagen, type XI, alpha 1 | 241 |
| PAPPA | pregnancy-associated plasma protein A, | 237 |
| | pappalysin 1 | |
| UCHL1 | ubiquitin carboxyl-terminal esterase L1 | 229 |
| SCG5 | secretogranin V (7B2 protein) | 218 |
| DKK1 | dickkopf homolog 1 | 193 |
| MICAL2 | microtubule associated monoxygenase, | 190 |
| | calponin and LIM domain 2 | |
| CDH2 | cadherin 2, type 1, N-cadherin | 175 |
| GREM2 | gremlin 2, | 163 |
| FN1 | fibronectin 1 | 160 |
| FOXD1 | forkhead box D1 | 151 |
| GREM1 | gremlin 1, | 140 |
| TRIB3 | tribbles homolog 3 | 136 |
| POPDC3 | popeye domain containing 3 | 126 |
| TMEM158 | transmembrane protein 158 | 124 |
| SCD | stearoyl-CoA desaturase | 124 |
| CNIH3 | cornichon homolog 3 | 122 |
| ELTD1 | EGF, latrophilin and seven transmembrane | 116 |
| | domain 1 | |
| FADS1 | fatty acid desaturase 1 | 110 |
| LTBP1 | latent transforming growth factor beta binding | 106 |
| | protein 1 | |
| ITGA10 | integrin, alpha 10 | 105 |

TABLE 4
Genes upregulated in uncultured AT-MSC compared with AT-MSC
expanded in monolayer and then entrapped in alginate.
Selection criteria: p < 0.01, top 30 genes by fold change

| | | Fold |
|---|---|---|
| Symbol | Description | change |
| CXCL14 | chemokine (C—X—C motif) ligand 14 | 6841 |
| CHRDL1 | chordin-like 1 | 3304 |
| CFD | complement factor D (adipsin) | 3019 |
| ADH1B | alcohol dehydrogenase IB, beta | 2978 |
| APOD | apolipoprotein D | 2937 |
| SPARCL1 | SPARC-like 1 (hevin) | 2521 |
| SEPP1 | selenoprotein P, plasma, 1 | 2320 |
| ITIH5 | inter-alpha (globulin) inhibitor H5 | 2180 |
| FABP4 | fatty acid binding protein 4, | 2020 |
| C7 | complement component 7 | 1438 |
| FMO2 | flavin containing monooxygenase 2 | 1252 |
| PDGFRL | platelet-derived growth factor receptor-like | 1235 |
| ITM2A | integral membrane protein 2A | 1193 |
| CHL1 | cell adhesion molecule with homology to L1CAM | 1184 |
| CILP | cartilage intermediate layer protein | 1160 |
| MYOC | myocilin | 1136 |
| NTRK2 | neurotrophic tyrosine kinase, receptor, type 2 | 1082 |
| LPL | lipoprotein lipase | 982 |
| SERPINA3 | serpin peptidase inhibitor, clade A, 3 | 976 |
| AADAC | arylacetamide deacetylase | 885 |
| CLEC3B | C-type lectin domain family 3, B | 676 |
| SPRY1 | sprouty homolog 1, antagonist of FGF signaling | 644 |
| RGS5 | regulator of G-protein signalling 5 | 556 |
| FMO1 | flavin containing monooxygenase 1 | 501 |
| WNT11 | wingless-type MMTV integration site family, 11 | 468 |
| PPL | periplakin | 452 |
| OMD | osteomodulin | 422 |
| OGN | osteoglycin (mimecan) | 402 |
| TNFSF10 | tumor necrosis factor (ligand) superfamily, 10 | 360 |
| MATN2 | matrilin 2 | 357 |

SUPPLEMENTAL TABLE 1
Gene ontology terms in the list with p value of less than 0.05, for upregulated in RGD vs Monolayer

| | | | | % of Genes in | |
|---|---|---|---|---|---|
| | Genes in | % of Genes | Genes in List | List in | |
| Upregulated RGD vs monolayer | Category | in Category | in Category | Category | p-Value |
| GO: 7160: cell-matrix adhesion | 143 | 0.838 | 4 | 9.756 | 0.000376 |
| GO: 31589: cell-substrate adhesion | 145 | 0.849 | 4 | 9.756 | 0.000396 |
| GO: 15804: neutral amino acid transport | 19 | 0.111 | 2 | 4.878 | 0.000938 |
| GO: 7229: integrin-mediated signaling | 102 | 0.598 | 3 | 7.317 | 0.00187 |
| pathway | | | | | |
| GO: 15807: L-amino acid transport | 29 | 0.17 | 2 | 4.878 | 0.00219 |
| GO: 1510: RNA methylation | 2 | 0.0117 | 1 | 2.439 | 0.0048 |
| GO: 7596: blood coagulation | 148 | 0.867 | 3 | 7.317 | 0.00535 |
| GO: 50817: coagulation | 152 | 0.891 | 3 | 7.317 | 0.00576 |
| GO: 7599: hemostasis | 157 | 0.92 | 3 | 7.317 | 0.0063 |
| GO: 7338: fertilization (sensu Metazoa) | 57 | 0.334 | 2 | 4.878 | 0.00826 |
| GO: 50878: regulation of body fluids | 174 | 1.019 | 3 | 7.317 | 0.00835 |
| GO: 9566: fertilization | 58 | 0.34 | 2 | 4.878 | 0.00855 |
| GO: 6865: amino acid transport | 60 | 0.352 | 2 | 4.878 | 0.00912 |
| GO: 45210: FasL biosynthesis | 4 | 0.0234 | 1 | 2.439 | 0.00957 |
| GO: 15014: heparan sulfate | 4 | 0.0234 | 1 | 2.439 | 0.00957 |
| proteoglycan biosynthesis, | | | | | |
| polysaccharide chain biosynthesis | | | | | |
| GO: 42060: wound healing | 185 | 1.084 | 3 | 7.317 | 0.00987 |
| GO: 7155: cell adhesion | 1051 | 6.157 | 7 | 17.07 | 0.0117 |
| GO: 31017: exocrine pancreas | 5 | 0.0293 | 1 | 2.439 | 0.012 |
| development | | | | | |
| GO: 30202: heparin metabolism | 5 | 0.0293 | 1 | 2.439 | 0.012 |
| GO: 9308: amine metabolism | 587 | 3.439 | 5 | 12.2 | 0.0128 |
| GO: 15837: amine transport | 79 | 0.463 | 2 | 4.878 | 0.0154 |
| GO: 6568: tryptophan metabolism | 7 | 0.041 | 1 | 2.439 | 0.0167 |
| GO: 6807: nitrogen compound | 630 | 3.691 | 5 | 12.2 | 0.0169 |
| metabolism | | | | | |
| GO: 15849: organic acid transport | 96 | 0.562 | 2 | 4.878 | 0.0223 |
| GO: 46942: carboxylic acid transport | 96 | 0.562 | 2 | 4.878 | 0.0223 |
| GO: 6043: glucosamine catabolism | 10 | 0.0586 | 1 | 2.439 | 0.0238 |
| GO: 46348: amino sugar catabolism | 10 | 0.0586 | 1 | 2.439 | 0.0238 |
| GO: 45598: regulation of fat cell | 11 | 0.0644 | 1 | 2.439 | 0.0261 |
| differentiation | | | | | |
| GO: 1504: neurotransmitter uptake | 12 | 0.0703 | 1 | 2.439 | 0.0285 |
| GO: 15012: heparan sulfate | 13 | 0.0762 | 1 | 2.439 | 0.0308 |
| proteoglycan biosynthesis | | | | | |
| GO: 1505: regulation of | 116 | 0.68 | 2 | 4.878 | 0.0316 |
| neurotransmitter levels | | | | | |
| GO: 6586: indolalkylamine metabolism | 15 | 0.0879 | 1 | 2.439 | 0.0354 |
| GO: 42430: indole and derivative | 15 | 0.0879 | 1 | 2.439 | 0.0354 |
| metabolism | | | | | |
| GO: 42434: indole derivative | 15 | 0.0879 | 1 | 2.439 | 0.0354 |
| metabolism | | | | | |
| GO: 7044: cell-substrate junction | 15 | 0.0879 | 1 | 2.439 | 0.0354 |
| assembly | | | | | |
| GO: 30201: heparan sulfate | 16 | 0.0937 | 1 | 2.439 | 0.0378 |
| proteoglycan metabolism | | | | | |
| GO: 50931: pigment cell differentiation | 18 | 0.105 | 1 | 2.439 | 0.0424 |
| GO: 30318: melanocyte differentiation | 18 | 0.105 | 1 | 2.439 | 0.0424 |
| GO: 31016: pancreas development | 20 | 0.117 | 1 | 2.439 | 0.047 |

SUPPLEMENTAL TABLE 2
Gene ontology terms in the list with p value of less than 0.05,
for upregulated in monolayer vs RGD

| | | | Genes in | % of Genes | |
|---|---|---|---|---|---|
| | Genes in | % of Genes | list in | in List in | |
| Upregulated monolayer vs RGD | category | in Category | category | Category | p-Value |
| GO: 8360: regulation of cell shape | 74 | 0.434 | 3 | 8.571 | 0.000463 |
| GO: 9312: oligosaccharide | 16 | 0.0937 | 2 | 5.714 | 0.000481 |
| biosynthesis | | | | | |
| GO: 9311: oligosaccharide | 34 | 0.199 | 2 | 5.714 | 0.0022 |
| metabolism | | | | | |
| GO: 50779: RNA destabilization | 3 | 0.0176 | 1 | 2.857 | 0.00614 |
| GO: 7265: Ras protein signal | 91 | 0.533 | 2 | 5.714 | 0.0149 |
| transduction | | | | | |
| GO: 902: cellular morphogenesis | 720 | 4.218 | 5 | 14.29 | 0.015 |
| GO: 31032: actomyosin structure | 8 | 0.0469 | 1 | 2.857 | 0.0163 |
| organization and biogenesis | | | | | |
| GO: 48535: lymph node | 11 | 0.0644 | 1 | 2.857 | 0.0223 |
| development | | | | | |
| GO: 7565: pregnancy | 123 | 0.721 | 2 | 5.714 | 0.0262 |
| GO: 6368: RNA elongation from | 13 | 0.0762 | 1 | 2.857 | 0.0263 |
| RNA polymerase II promoter | | | | | |
| GO: 50728: negative regulation of | 13 | 0.0762 | 1 | 2.857 | 0.0263 |
| inflammatory response | | | | | |
| GO: 16051: carbohydrate | 130 | 0.762 | 2 | 5.714 | 0.0291 |
| biosynthesis | | | | | |
| GO: 7242: intracellular signaling | 1845 | 10.81 | 8 | 22.86 | 0.0302 |
| cascade | | | | | |
| GO: 7275: development | 3816 | 22.36 | 13 | 37.14 | 0.0339 |
| GO: 6354: RNA elongation | 17 | 0.0996 | 1 | 2.857 | 0.0343 |
| GO: 6144: purine base metabolism | 17 | 0.0996 | 1 | 2.857 | 0.0343 |
| GO: 6309: DNA fragmentation | 18 | 0.105 | 1 | 2.857 | 0.0363 |
| during apoptosis | | | | | |
| GO: 51291: protein | 18 | 0.105 | 1 | 2.857 | 0.0363 |
| heterooligomerization | | | | | |
| GO: 18: regulation of DNA | 19 | 0.111 | 1 | 2.857 | 0.0383 |
| recombination | | | | | |
| GO: 46330: positive regulation of | 19 | 0.111 | 1 | 2.857 | 0.0383 |
| JNK cascade | | | | | |
| GO: 45638: negative regulation of | 22 | 0.129 | 1 | 2.857 | 0.0442 |
| myeloid cell differentiation | | | | | |
| GO: 6486: protein amino acid | 167 | 0.978 | 2 | 5.714 | 0.0459 |
| glycosylation | | | | | |
| GO: 43413: biopolymer | 169 | 0.99 | 2 | 5.714 | 0.0469 |
| glycosylation | | | | | |
| GO: 7016: cytoskeletal anchoring | 24 | 0.141 | 1 | 2.857 | 0.0481 |

SUPPLEMENTAL TABLE 3
Gene ontology terms in the list with p value of less than 0.05,
for upregulated in RGD vs uncultured

| | | | | % of | |
|---|---|---|---|---|---|
| | | | Genes in | Genes in | |
| | Genes in | % of Genes | List in | List in | |
| Category | Category | in Category | Category | Category | p-Value |
| GO: 9058: biosynthesis | 1763 | 10.33 | 106 | 19.78 | 2.66E−11 |
| GO: 16126: sterol biosynthesis | 52 | 0.305 | 13 | 2.425 | 5.17E−09 |
| GO: 6096: glycolysis | 85 | 0.498 | 15 | 2.799 | 5.56E−08 |
| GO: 6091: generation of precursor | 791 | 4.634 | 54 | 10.07 | 6.68E−08 |
| metabolites and energy | | | | | |
| GO: 6066: alcohol metabolism | 443 | 2.595 | 36 | 6.716 | 1.98E−07 |
| GO: 6520: amino acid metabolism | 387 | 2.267 | 33 | 6.157 | 2.15E−07 |
| GO: 6865: amino acid transport | 60 | 0.352 | 12 | 2.239 | 2.88E−07 |
| GO: 6519: amino acid and derivative | 485 | 2.841 | 37 | 6.903 | 6.36E−07 |
| metabolism | | | | | |
| GO: 6092: main pathways of | 177 | 1.037 | 20 | 3.731 | 7.70E−07 |
| carbohydrate metabolism | | | | | |
| GO: 6007: glucose catabolism | 104 | 0.609 | 15 | 2.799 | 8.52E−07 |
| GO: 19752: carboxylic acid | 736 | 4.312 | 48 | 8.955 | 1.39E−06 |
| metabolism | | | | | |
| GO: 6694: steroid biosynthesis | 108 | 0.633 | 15 | 2.799 | 1.39E−06 |
| GO: 6082: organic acid metabolism | 738 | 4.324 | 48 | 8.955 | 1.50E−06 |
| GO: 6807: nitrogen compound | 630 | 3.691 | 43 | 8.022 | 1.57E−06 |
| metabolism | | | | | |
| GO: 44249: cellular biosynthesis | 1567 | 9.18 | 82 | 15.3 | 2.59E−06 |
| GO: 6695: cholesterol biosynthesis | 40 | 0.234 | 9 | 1.679 | 3.18E−06 |
| GO: 44262: cellular carbohydrate | 499 | 2.923 | 36 | 6.716 | 3.30E−06 |
| metabolism | | | | | |
| GO: 9308: amine metabolism | 587 | 3.439 | 40 | 7.463 | 3.81E−06 |
| GO: 9259: ribonucleotide metabolism | 133 | 0.779 | 16 | 2.985 | 4.31E−06 |
| GO: 46365: monosaccharide | 121 | 0.709 | 15 | 2.799 | 5.90E−06 |
| catabolism | | | | | |
| GO: 19320: hexose catabolism | 121 | 0.709 | 15 | 2.799 | 5.90E−06 |
| GO: 8610: lipid biosynthesis | 330 | 1.933 | 27 | 5.037 | 5.96E−06 |
| GO: 15837: amine transport | 79 | 0.463 | 12 | 2.239 | 6.13E−06 |
| GO: 46164: alcohol catabolism | 124 | 0.726 | 15 | 2.799 | 8.00E−06 |
| GO: 15849: organic acid transport | 96 | 0.562 | 13 | 2.425 | 9.30E−06 |
| GO: 46942: carboxylic acid transport | 96 | 0.562 | 13 | 2.425 | 9.30E−06 |
| GO: 6163: purine nucleotide | 126 | 0.738 | 15 | 2.799 | 9.74E−06 |
| metabolism | | | | | |
| GO: 43038: amino acid activation | 58 | 0.34 | 10 | 1.866 | 1.15E−05 |
| GO: 43039: tRNA aminoacylation | 58 | 0.34 | 10 | 1.866 | 1.15E−05 |
| GO: 6418: tRNA aminoacylation for | 58 | 0.34 | 10 | 1.866 | 1.15E−05 |
| protein translation | | | | | |
| GO: 19318: hexose metabolism | 231 | 1.353 | 21 | 3.918 | 1.34E−05 |
| GO: 15980: energy derivation by | 268 | 1.57 | 23 | 4.291 | 1.35E−05 |
| oxidation of organic compounds | | | | | |
| GO: 9165: nucleotide biosynthesis | 196 | 1.148 | 19 | 3.545 | 1.39E−05 |
| GO: 6006: glucose metabolism | 165 | 0.967 | 17 | 3.172 | 1.77E−05 |
| GO: 5996: monosaccharide | 236 | 1.383 | 21 | 3.918 | 1.85E−05 |
| metabolism | | | | | |
| GO: 9260: ribonucleotide biosynthesis | 118 | 0.691 | 14 | 2.612 | 2.00E−05 |
| GO: 9150: purine ribonucleotide | 119 | 0.697 | 14 | 2.612 | 2.20E−05 |
| metabolism | | | | | |
| GO: 16052: carbohydrate catabolism | 152 | 0.891 | 16 | 2.985 | 2.39E−05 |
| GO: 44275: cellular carbohydrate | 152 | 0.891 | 16 | 2.985 | 2.39E−05 |
| catabolism | | | | | |
| GO: 5975: carbohydrate metabolism | 637 | 3.732 | 40 | 7.463 | 2.58E−05 |
| GO: 51089: constitutive protein | 3 | 0.0176 | 3 | 0.56 | 3.08E−05 |
| ectodomain proteolysis | | | | | |
| GO: 51186: cofactor metabolism | 267 | 1.564 | 22 | 4.104 | 3.86E−05 |
| GO: 6164: purine nucleotide | 112 | 0.656 | 13 | 2.425 | 4.96E−05 |
| biosynthesis | | | | | |
| GO: 6457: protein folding | 341 | 1.998 | 25 | 4.664 | 8.08E−05 |
| GO: 9152: purine ribonucleotide | 106 | 0.621 | 12 | 2.239 | 0.000122 |
| biosynthesis | | | | | |
| GO: 6100: tricarboxylic acid cycle | 37 | 0.217 | 7 | 1.306 | 0.000131 |
| intermediate metabolism | | | | | |
| GO: 6732: coenzyme metabolism | 216 | 1.265 | 18 | 3.358 | 0.000167 |
| GO: 9199: ribonucleoside triphosphate | 96 | 0.562 | 11 | 2.052 | 0.000209 |
| metabolism | | | | | |
| GO: 16125: sterol metabolism | 130 | 0.762 | 13 | 2.425 | 0.000229 |
| GO: 9117: nucleotide metabolism | 302 | 1.769 | 22 | 4.104 | 0.000233 |
| GO: 15807: L-amino acid transport | 29 | 0.17 | 6 | 1.119 | 0.000239 |
| GO: 44248: cellular catabolism | 803 | 4.704 | 44 | 8.209 | 0.000243 |
| GO: 9141: nucleoside triphosphate | 103 | 0.603 | 11 | 2.052 | 0.000388 |
| metabolism | | | | | |
| GO: 9991: response to extracellular | 45 | 0.264 | 7 | 1.306 | 0.000466 |
| stimulus | | | | | |
| GO: 43037: translation | 219 | 1.283 | 17 | 3.172 | 0.000571 |
| GO: 44265: cellular macromolecule | 508 | 2.976 | 30 | 5.597 | 0.000728 |
| catabolism | | | | | |
| GO: 9205: purine ribonucleoside | 95 | 0.557 | 10 | 1.866 | 0.000792 |
| triphosphate metabolism | | | | | |
| GO: 9144: purine nucleoside | 96 | 0.562 | 10 | 1.866 | 0.00086 |
| triphosphate metabolism | | | | | |
| GO: 6541: glutamine metabolism | 25 | 0.146 | 5 | 0.933 | 0.000945 |
| GO: 7412: axon target recognition | 2 | 0.0117 | 2 | 0.373 | 0.000984 |
| GO: 6478: peptidyl-tyrosine sulfation | 2 | 0.0117 | 2 | 0.373 | 0.000984 |
| GO: 19255: glucose 1-phosphate | 2 | 0.0117 | 2 | 0.373 | 0.000984 |
| metabolism | | | | | |
| GO: 9056: catabolism | 926 | 5.425 | 46 | 8.582 | 0.00142 |
| GO: 6636: fatty acid desaturation | 8 | 0.0469 | 3 | 0.56 | 0.00153 |
| GO: 8202: steroid metabolism | 261 | 1.529 | 18 | 3.358 | 0.00156 |
| GO: 31667: response to nutrient levels | 41 | 0.24 | 6 | 1.119 | 0.00165 |
| GO: 6399: tRNA metabolism | 105 | 0.615 | 10 | 1.866 | 0.00171 |
| GO: 46034: ATP metabolism | 73 | 0.428 | 8 | 1.493 | 0.00201 |
| GO: 46483: heterocycle metabolism | 109 | 0.639 | 10 | 1.866 | 0.00226 |
| GO: 6953: acute-phase response | 44 | 0.258 | 6 | 1.119 | 0.00239 |
| GO: 9064: glutamine family amino | 60 | 0.352 | 7 | 1.306 | 0.00265 |
| acid metabolism | | | | | |
| GO: 6431: methionyl-tRNA | 3 | 0.0176 | 2 | 0.373 | 0.00289 |
| aminoacylation | | | | | |
| GO: 6436: tryptophanyl-tRNA | 3 | 0.0176 | 2 | 0.373 | 0.00289 |
| aminoacylation | | | | | |
| GO: 9207: purine ribonucleoside | 3 | 0.0176 | 2 | 0.373 | 0.00289 |
| triphosphate catabolism | | | | | |
| GO: 6200: ATP catabolism | 3 | 0.0176 | 2 | 0.373 | 0.00289 |
| GO: 9203: ribonucleoside triphosphate | 3 | 0.0176 | 2 | 0.373 | 0.00289 |
| catabolism | | | | | |
| GO: 6741: NADP biosynthesis | 3 | 0.0176 | 2 | 0.373 | 0.00289 |
| GO: 101: sulfur amino acid transport | 3 | 0.0176 | 2 | 0.373 | 0.00289 |
| GO: 15811: L-cystine transport | 3 | 0.0176 | 2 | 0.373 | 0.00289 |
| GO: 6188: IMP biosynthesis | 10 | 0.0586 | 3 | 0.56 | 0.00313 |
| GO: 6189: ‘de novo’ IMP biosynthesis | 10 | 0.0586 | 3 | 0.56 | 0.00313 |
| GO: 6108: malate metabolism | 10 | 0.0586 | 3 | 0.56 | 0.00313 |
| GO: 46040: IMP metabolism | 10 | 0.0586 | 3 | 0.56 | 0.00313 |
| GO: 31669: cellular response to | 10 | 0.0586 | 3 | 0.56 | 0.00313 |
| nutrient levels | | | | | |
| GO: 9267: cellular response to | 10 | 0.0586 | 3 | 0.56 | 0.00313 |
| starvation | | | | | |
| GO: 31668: cellular response to | 10 | 0.0586 | 3 | 0.56 | 0.00313 |
| extracellular stimulus | | | | | |
| GO: 9057: macromolecule catabolism | 560 | 3.281 | 30 | 5.597 | 0.00323 |
| GO: 6221: pyrimidine nucleotide | 34 | 0.199 | 5 | 0.933 | 0.00393 |
| biosynthesis | | | | | |
| GO: 9124: nucleoside monophosphate | 34 | 0.199 | 5 | 0.933 | 0.00393 |
| biosynthesis | | | | | |
| GO: 9123: nucleoside monophosphate | 34 | 0.199 | 5 | 0.933 | 0.00393 |
| metabolism | | | | | |
| GO: 51270: regulation of cell motility | 100 | 0.586 | 9 | 1.679 | 0.0042 |
| GO: 42594: response to starvation | 11 | 0.0644 | 3 | 0.56 | 0.00421 |
| GO: 7162: negative regulation of cell | 35 | 0.205 | 5 | 0.933 | 0.00446 |
| adhesion | | | | | |
| GO: 45454: cell redox homeostasis | 66 | 0.387 | 7 | 1.306 | 0.00455 |
| GO: 51188: cofactor biosynthesis | 140 | 0.82 | 11 | 2.052 | 0.00468 |
| GO: 9201: ribonucleoside triphosphate | 84 | 0.492 | 8 | 1.493 | 0.00484 |
| biosynthesis | | | | | |
| GO: 42364: water-soluble vitamin | 23 | 0.135 | 4 | 0.746 | 0.0053 |
| biosynthesis | | | | | |
| GO: 6118: electron transport | 434 | 2.543 | 24 | 4.478 | 0.00546 |
| GO: 9113: purine base biosynthesis | 12 | 0.0703 | 3 | 0.56 | 0.00548 |
| GO: 9142: nucleoside triphosphate | 86 | 0.504 | 8 | 1.493 | 0.00558 |
| biosynthesis | | | | | |
| GO: 19471: 4-hydroxyproline | 4 | 0.0234 | 2 | 0.373 | 0.00566 |
| metabolism | | | | | |
| GO: 18401: peptidyl-proline | 4 | 0.0234 | 2 | 0.373 | 0.00566 |
| hydroxylation to 4-hydroxy-L-proline | | | | | |
| GO: 9146: purine nucleoside | 4 | 0.0234 | 2 | 0.373 | 0.00566 |
| triphosphate catabolism | | | | | |
| GO: 45210: FasL biosynthesis | 4 | 0.0234 | 2 | 0.373 | 0.00566 |
| GO: 6101: citrate metabolism | 4 | 0.0234 | 2 | 0.373 | 0.00566 |
| GO: 19511: peptidyl-proline | 4 | 0.0234 | 2 | 0.373 | 0.00566 |
| hydroxylation | | | | | |
| GO: 30334: regulation of cell | 87 | 0.51 | 8 | 1.493 | 0.00598 |
| migration | | | | | |
| GO: 6029: proteoglycan metabolism | 38 | 0.223 | 5 | 0.933 | 0.00639 |
| GO: 6986: response to unfolded | 89 | 0.521 | 8 | 1.493 | 0.00684 |
| protein | | | | | |
| GO: 9059: macromolecule | 1034 | 6.058 | 47 | 8.769 | 0.00692 |
| biosynthesis | | | | | |
| GO: 40012: regulation of locomotion | 108 | 0.633 | 9 | 1.679 | 0.00694 |
| GO: 50795: regulation of behavior | 108 | 0.633 | 9 | 1.679 | 0.00694 |
| GO: 7220: Notch receptor processing | 13 | 0.0762 | 3 | 0.56 | 0.00696 |
| GO: 9110: vitamin biosynthesis | 25 | 0.146 | 4 | 0.746 | 0.0072 |
| GO: 6509: membrane protein | 25 | 0.146 | 4 | 0.746 | 0.0072 |
| ectodomain proteolysis | | | | | |
| GO: 6725: aromatic compound | 174 | 1.019 | 12 | 2.239 | 0.00895 |
| metabolism | | | | | |
| GO: 9143: nucleoside triphosphate | 5 | 0.0293 | 2 | 0.373 | 0.00924 |
| catabolism | | | | | |
| GO: 18208: peptidyl-proline | 5 | 0.0293 | 2 | 0.373 | 0.00924 |
| modification | | | | | |
| GO: 320: re-entry into mitotic cell | 5 | 0.0293 | 2 | 0.373 | 0.00924 |
| cycle | | | | | |
| GO: 51234: establishment of | 4175 | 24.46 | 155 | 28.92 | 0.00929 |
| localization | | | | | |
| GO: 1502: cartilage condensation | 27 | 0.158 | 4 | 0.746 | 0.00951 |
| GO: 9220: pyrimidine ribonucleotide | 27 | 0.158 | 4 | 0.746 | 0.00951 |
| biosynthesis | | | | | |
| GO: 19363: pyridine nucleotide | 15 | 0.0879 | 3 | 0.56 | 0.0106 |
| biosynthesis | | | | | |
| GO: 51179: localization | 4235 | 24.81 | 156 | 29.1 | 0.012 |
| GO: 9218: pyrimidine ribonucleotide | 29 | 0.17 | 4 | 0.746 | 0.0122 |
| metabolism | | | | | |
| GO: 9108: coenzyme biosynthesis | 119 | 0.697 | 9 | 1.679 | 0.0127 |
| GO: 8203: cholesterol metabolism | 119 | 0.697 | 9 | 1.679 | 0.0127 |
| GO: 9310: amine catabolism | 99 | 0.58 | 8 | 1.493 | 0.0127 |
| GO: 30201: heparan sulfate | 16 | 0.0937 | 3 | 0.56 | 0.0127 |
| proteoglycan metabolism | | | | | |
| GO: 30968: unfolded protein response | 16 | 0.0937 | 3 | 0.56 | 0.0127 |
| GO: 6752: group transfer coenzyme | 81 | 0.475 | 7 | 1.306 | 0.0136 |
| metabolism | | | | | |
| GO: 9263: deoxyribonucleotide | 6 | 0.0352 | 2 | 0.373 | 0.0136 |
| biosynthesis | | | | | |
| GO: 6002: fructose 6-phosphate | 6 | 0.0352 | 2 | 0.373 | 0.0136 |
| metabolism | | | | | |
| GO: 44270: nitrogen compound | 101 | 0.592 | 8 | 1.493 | 0.0142 |
| catabolism | | | | | |
| GO: 7229: integrin-mediated signaling | 102 | 0.598 | 8 | 1.493 | 0.015 |
| pathway | | | | | |
| GO: 6144: purine base metabolism | 17 | 0.0996 | 3 | 0.56 | 0.0151 |
| GO: 9063: amino acid catabolism | 83 | 0.486 | 7 | 1.306 | 0.0154 |
| GO: 9145: purine nucleoside | 83 | 0.486 | 7 | 1.306 | 0.0154 |
| triphosphate biosynthesis | | | | | |
| GO: 9206: purine ribonucleoside | 83 | 0.486 | 7 | 1.306 | 0.0154 |
| triphosphate biosynthesis | | | | | |
| GO: 9072: aromatic amino acid family | 31 | 0.182 | 4 | 0.746 | 0.0155 |
| metabolism | | | | | |
| GO: 9156: ribonucleoside | 31 | 0.182 | 4 | 0.746 | 0.0155 |
| monophosphate biosynthesis | | | | | |
| GO: 9161: ribonucleoside | 31 | 0.182 | 4 | 0.746 | 0.0155 |
| monophosphate metabolism | | | | | |
| GO: 6769: nicotinamide metabolism | 32 | 0.187 | 4 | 0.746 | 0.0172 |
| GO: 45620: negative regulation of | 7 | 0.041 | 2 | 0.373 | 0.0186 |
| lymphocyte differentiation | | | | | |
| GO: 9154: purine ribonucleotide | 7 | 0.041 | 2 | 0.373 | 0.0186 |
| catabolism | | | | | |
| GO: 6979: response to oxidative stress | 87 | 0.51 | 7 | 1.306 | 0.0195 |
| GO: 51084: posttranslational protein | 19 | 0.111 | 3 | 0.56 | 0.0205 |
| folding | | | | | |
| GO: 15804: neutral amino acid | 19 | 0.111 | 3 | 0.56 | 0.0205 |
| transport | | | | | |
| GO: 7155: cell adhesion | 1051 | 6.157 | 45 | 8.396 | 0.0214 |
| GO: 6888: ER to Golgi transport | 130 | 0.762 | 9 | 1.679 | 0.0215 |
| GO: 9112: nucleobase metabolism | 35 | 0.205 | 4 | 0.746 | 0.0233 |
| GO: 9209: pyrimidine ribonucleoside | 20 | 0.117 | 3 | 0.56 | 0.0236 |
| triphosphate biosynthesis | | | | | |
| GO: 6241: CTP biosynthesis | 20 | 0.117 | 3 | 0.56 | 0.0236 |
| GO: 46112: nucleobase biosynthesis | 20 | 0.117 | 3 | 0.56 | 0.0236 |
| GO: 9208: pyrimidine ribonucleoside | 20 | 0.117 | 3 | 0.56 | 0.0236 |
| triphosphate metabolism | | | | | |
| GO: 46036: CTP metabolism | 20 | 0.117 | 3 | 0.56 | 0.0236 |
| GO: 6984: ER-nuclear signaling | 20 | 0.117 | 3 | 0.56 | 0.0236 |
| pathway | | | | | |
| GO: 6195: purine nucleotide | 8 | 0.0469 | 2 | 0.373 | 0.0243 |
| catabolism | | | | | |
| GO: 6220: pyrimidine nucleotide | 53 | 0.311 | 5 | 0.933 | 0.025 |
| metabolism | | | | | |
| GO: 19362: pyridine nucleotide | 36 | 0.211 | 4 | 0.746 | 0.0256 |
| metabolism | | | | | |
| GO: 9127: purine nucleoside | 21 | 0.123 | 3 | 0.56 | 0.0269 |
| monophosphate biosynthesis | | | | | |
| GO: 9168: purine ribonucleoside | 21 | 0.123 | 3 | 0.56 | 0.0269 |
| monophosphate biosynthesis | | | | | |
| GO: 9126: purine nucleoside | 21 | 0.123 | 3 | 0.56 | 0.0269 |
| monophosphate metabolism | | | | | |
| GO: 9167: purine ribonucleoside | 21 | 0.123 | 3 | 0.56 | 0.0269 |
| monophosphate metabolism | | | | | |
| GO: 6790: sulfur metabolism | 94 | 0.551 | 7 | 1.306 | 0.0284 |
| GO: 6800: oxygen and reactive | 116 | 0.68 | 8 | 1.493 | 0.0298 |
| oxygen species metabolism | | | | | |
| GO: 9636: response to toxin | 22 | 0.129 | 3 | 0.56 | 0.0304 |
| GO: 46907: intracellular transport | 1021 | 5.982 | 43 | 8.022 | 0.0306 |
| GO: 19627: urea metabolism | 9 | 0.0527 | 2 | 0.373 | 0.0306 |
| GO: 50: urea cycle | 9 | 0.0527 | 2 | 0.373 | 0.0306 |
| GO: 6702: androgen biosynthesis | 9 | 0.0527 | 2 | 0.373 | 0.0306 |
| GO: 15813: L-glutamate transport | 9 | 0.0527 | 2 | 0.373 | 0.0306 |
| GO: 19748: secondary metabolism | 56 | 0.328 | 5 | 0.933 | 0.0308 |
| GO: 7406: negative regulation of | 1 | 0.00586 | 1 | 0.187 | 0.0314 |
| neuroblast proliferation | | | | | |
| GO: 6437: tyrosyl-tRNA | 1 | 0.00586 | 1 | 0.187 | 0.0314 |
| aminoacylation | | | | | |
| GO: 6172: ADP biosynthesis | 1 | 0.00586 | 1 | 0.187 | 0.0314 |
| GO: 9183: purine deoxyribonucleoside | 1 | 0.00586 | 1 | 0.187 | 0.0314 |
| diphosphate biosynthesis | | | | | |
| GO: 6173: dADP biosynthesis | 1 | 0.00586 | 1 | 0.187 | 0.0314 |
| GO: 9153: purine deoxyribonucleotide | 1 | 0.00586 | 1 | 0.187 | 0.0314 |
| biosynthesis | | | | | |
| GO: 51045: negative regulation of | 1 | 0.00586 | 1 | 0.187 | 0.0314 |
| membrane protein ectodomain | | | | | |
| proteolysis | | | | | |
| GO: 51043: regulation of membrane | 1 | 0.00586 | 1 | 0.187 | 0.0314 |
| protein ectodomain proteolysis | | | | | |
| GO: 31639: plasminogen activation | 1 | 0.00586 | 1 | 0.187 | 0.0314 |
| GO: 42262: DNA protection | 1 | 0.00586 | 1 | 0.187 | 0.0314 |
| GO: 9182: purine deoxyribonucleoside | 1 | 0.00586 | 1 | 0.187 | 0.0314 |
| diphosphate metabolism | | | | | |
| GO: 46056: dADP metabolism | 1 | 0.00586 | 1 | 0.187 | 0.0314 |
| GO: 7035: vacuolar acidification | 1 | 0.00586 | 1 | 0.187 | 0.0314 |
| GO: 15822: L-ornithine transport | 1 | 0.00586 | 1 | 0.187 | 0.0314 |
| GO: 66: mitochondrial ornithine | 1 | 0.00586 | 1 | 0.187 | 0.0314 |
| transport | | | | | |
| GO: 44255: cellular lipid metabolism | 778 | 4.558 | 34 | 6.343 | 0.0327 |
| GO: 15986: ATP synthesis coupled | 58 | 0.34 | 5 | 0.933 | 0.0351 |
| proton transport | | | | | |
| GO: 15985: energy coupled proton | 58 | 0.34 | 5 | 0.933 | 0.0351 |
| transport, down electrochemical | | | | | |
| gradient | | | | | |
| GO: 46209: nitric oxide metabolism | 40 | 0.234 | 4 | 0.746 | 0.036 |
| GO: 6809: nitric oxide biosynthesis | 40 | 0.234 | 4 | 0.746 | 0.036 |
| GO: 8037: cell recognition | 40 | 0.234 | 4 | 0.746 | 0.036 |
| GO: 6527: arginine catabolism | 10 | 0.0586 | 2 | 0.373 | 0.0375 |
| GO: 9261: ribonucleotide catabolism | 10 | 0.0586 | 2 | 0.373 | 0.0375 |
| GO: 15936: coenzyme A metabolism | 10 | 0.0586 | 2 | 0.373 | 0.0375 |
| GO: 15800: acidic amino acid | 10 | 0.0586 | 2 | 0.373 | 0.0375 |
| transport | | | | | |
| GO: 6739: NADP metabolism | 24 | 0.141 | 3 | 0.56 | 0.0382 |
| GO: 51649: establishment of cellular | 1039 | 6.087 | 43 | 8.022 | 0.039 |
| localization | | | | | |
| GO: 6412: protein biosynthesis | 928 | 5.437 | 39 | 7.276 | 0.0393 |
| GO: 6754: ATP biosynthesis | 61 | 0.357 | 5 | 0.933 | 0.0423 |
| GO: 6767: water-soluble vitamin | 61 | 0.357 | 5 | 0.933 | 0.0423 |
| metabolism | | | | | |
| GO: 6753: nucleoside phosphate | 61 | 0.357 | 5 | 0.933 | 0.0423 |
| metabolism | | | | | |
| GO: 9147: pyrimidine nucleoside | 25 | 0.146 | 3 | 0.56 | 0.0424 |
| triphosphate metabolism | | | | | |
| GO: 7271: synaptic transmission, | 25 | 0.146 | 3 | 0.56 | 0.0424 |
| cholinergic | | | | | |
| GO: 48193: Golgi vesicle transport | 195 | 1.142 | 11 | 2.052 | 0.0442 |
| GO: 6477: protein amino acid | 11 | 0.0644 | 2 | 0.373 | 0.0449 |
| sulfation | | | | | |
| GO: 6890: retrograde transport, Golgi | 26 | 0.152 | 3 | 0.56 | 0.0469 |
| to ER | | | | | |
| GO: 7052: mitotic spindle | 26 | 0.152 | 3 | 0.56 | 0.0469 |
| organization and biogenesis | | | | | |
| GO: 30261: chromosome | 26 | 0.152 | 3 | 0.56 | 0.0469 |
| condensation | | | | | |
| GO: 30178: negative regulation of | 26 | 0.152 | 3 | 0.56 | 0.0469 |
| Wnt receptor signaling pathway | | | | | |
| GO: 6810: transport | 3505 | 20.53 | 126 | 23.51 | 0.0484 |

SUPPLEMENTAL TABLE 4
Gene ontology terms in the list with p value of less
than 0.05, for upregulated in uncultured vs RGD

| | | % of | Genes in | % of Genes in | |
|---|---|---|---|---|---|
| Upregulated uncultured | Genes in | Genes in | List in | List in | |
| vs RGD | Category | Category | Category | Category | p-Value |
| GO: 7275: development | 3816 | 22.36 | 152 | 33.33 | 3.34E−08 |
| GO: 30154: cell | 1482 | 8.682 | 74 | 16.23 | 9.95E−08 |
| differentiation | | | | | |
| GO: 45637: regulation of | 69 | 0.404 | 11 | 2.412 | 1.98E−06 |
| myeloid cell | | | | | |
| differentiation | | | | | |
| GO: 30111: regulation of | 45 | 0.264 | 9 | 1.974 | 2.42E−06 |
| Wnt receptor signaling | | | | | |
| pathway | | | | | |
| GO: 48519: negative | 1841 | 10.79 | 80 | 17.54 | 7.41E−06 |
| regulation of biological | | | | | |
| process | | | | | |
| GO: 42127: regulation of | 730 | 4.277 | 40 | 8.772 | 1.42E−05 |
| cell proliferation | | | | | |
| GO: 7517: muscle | 276 | 1.617 | 21 | 4.605 | 1.77E−05 |
| development | | | | | |
| GO: 48513: organ | 1675 | 9.813 | 73 | 16.01 | 1.81E−05 |
| development | | | | | |
| GO: 35026: leading edge | 3 | 0.0176 | 3 | 0.658 | 1.89E−05 |
| cell differentiation | | | | | |
| GO: 30185: nitric oxide | 3 | 0.0176 | 3 | 0.658 | 1.89E−05 |
| transport | | | | | |
| GO: 9966: regulation of | 663 | 3.884 | 37 | 8.114 | 2.02E−05 |
| signal transduction | | | | | |
| GO: 30099: myeloid cell | 139 | 0.814 | 14 | 3.07 | 2.16E−05 |
| differentiation | | | | | |
| GO: 48523: negative | 1723 | 10.09 | 74 | 16.23 | 2.59E−05 |
| regulation of cellular | | | | | |
| process | | | | | |
| GO: 9653: morphogenesis | 1716 | 10.05 | 73 | 16.01 | 4.05E−05 |
| GO: 8593: regulation of | 16 | 0.0937 | 5 | 1.096 | 4.56E−05 |
| Notch signaling pathway | | | | | |
| GO: 45165: cell fate | 114 | 0.668 | 12 | 2.632 | 5.37E−05 |
| commitment | | | | | |
| GO: 6067: ethanol | 9 | 0.0527 | 4 | 0.877 | 5.69E−05 |
| metabolism | | | | | |
| GO: 6069: ethanol | 9 | 0.0527 | 4 | 0.877 | 5.69E−05 |
| oxidation | | | | | |
| GO: 185: activation of | 9 | 0.0527 | 4 | 0.877 | 5.69E−05 |
| MAPKKK activity | | | | | |
| GO: 40007: growth | 402 | 2.355 | 25 | 5.482 | 8.58E−05 |
| GO: 1709: cell fate | 44 | 0.258 | 7 | 1.535 | 0.000151 |
| determination | | | | | |
| GO: 45596: negative | 75 | 0.439 | 9 | 1.974 | 0.000169 |
| regulation of cell | | | | | |
| differentiation | | | | | |
| GO: 74: regulation of | 916 | 5.366 | 43 | 9.43 | 0.000239 |
| progression through cell | | | | | |
| cycle | | | | | |
| GO: 45638: negative | 22 | 0.129 | 5 | 1.096 | 0.000241 |
| regulation of myeloid cell | | | | | |
| differentiation | | | | | |
| GO: 9968: negative | 154 | 0.902 | 13 | 2.851 | 0.000255 |
| regulation of signal | | | | | |
| transduction | | | | | |
| GO: 6800: oxygen and | 116 | 0.68 | 11 | 2.412 | 0.000277 |
| reactive oxygen species | | | | | |
| metabolism | | | | | |
| GO: 8283: cell | 1199 | 7.024 | 52 | 11.4 | 0.00037 |
| proliferation | | | | | |
| GO: 6957: complement | 14 | 0.082 | 4 | 0.877 | 0.000407 |
| activation, alternative | | | | | |
| pathway | | | | | |
| GO: 6954: inflammatory | 335 | 1.963 | 20 | 4.386 | 0.000719 |
| response | | | | | |
| GO: 16055: Wnt receptor | 172 | 1.008 | 13 | 2.851 | 0.000737 |
| signaling pathway | | | | | |
| GO: 42551: neuron | 75 | 0.439 | 8 | 1.754 | 0.000859 |
| maturation | | | | | |
| GO: 45429: positive | 17 | 0.0996 | 4 | 0.877 | 0.000907 |
| regulation of nitric oxide | | | | | |
| biosynthesis | | | | | |
| GO: 51093: negative | 95 | 0.557 | 9 | 1.974 | 0.000987 |
| regulation of | | | | | |
| development | | | | | |
| GO: 48511: rhythmic | 96 | 0.562 | 9 | 1.974 | 0.00106 |
| process | | | | | |
| GO: 6633: fatty acid | 97 | 0.568 | 9 | 1.974 | 0.00115 |
| biosynthesis | | | | | |
| GO: 16049: cell growth | 299 | 1.752 | 18 | 3.947 | 0.00119 |
| GO: 7154: cell | 5403 | 31.65 | 175 | 38.38 | 0.00121 |
| communication | | | | | |
| GO: 8361: regulation of | 303 | 1.775 | 18 | 3.947 | 0.00138 |
| cell size | | | | | |
| GO: 48729: tissue | 82 | 0.48 | 8 | 1.754 | 0.00154 |
| morphogenesis | | | | | |
| GO: 6956: complement | 48 | 0.281 | 6 | 1.316 | 0.00167 |
| activation | | | | | |
| GO: 45670: regulation of | 20 | 0.117 | 4 | 0.877 | 0.00173 |
| osteoclast differentiation | | | | | |
| GO: 1501: skeletal | 335 | 1.963 | 19 | 4.167 | 0.00175 |
| development | | | | | |
| GO: 8285: negative | 361 | 2.115 | 20 | 4.386 | 0.00177 |
| regulation of cell | | | | | |
| proliferation | | | | | |
| GO: 48741: skeletal | 85 | 0.498 | 8 | 1.754 | 0.00195 |
| muscle fiber development | | | | | |
| GO: 48747: muscle fiber | 85 | 0.498 | 8 | 1.754 | 0.00195 |
| development | | | | | |
| GO: 45747: positive | 10 | 0.0586 | 3 | 0.658 | 0.00198 |
| regulation of Notch | | | | | |
| signaling pathway | | | | | |
| GO: 6982: response to | 3 | 0.0176 | 2 | 0.439 | 0.0021 |
| lipid hydroperoxide | | | | | |
| GO: 42749: regulation of | 3 | 0.0176 | 2 | 0.439 | 0.0021 |
| circadian sleep/wake | | | | | |
| cycle | | | | | |
| GO: 45187: regulation of | 3 | 0.0176 | 2 | 0.439 | 0.0021 |
| circadian sleep/wake | | | | | |
| cycle, sleep | | | | | |
| GO: 50802: circadian | 3 | 0.0176 | 2 | 0.439 | 0.0021 |
| sleep/wake cycle, sleep | | | | | |
| GO: 16053: organic acid | 106 | 0.621 | 9 | 1.974 | 0.00213 |
| biosynthesis | | | | | |
| GO: 46394: carboxylic | 106 | 0.621 | 9 | 1.974 | 0.00213 |
| acid biosynthesis | | | | | |
| GO: 79: regulation of | 69 | 0.404 | 7 | 1.535 | 0.0024 |
| cyclin dependent protein | | | | | |
| kinase activity | | | | | |
| GO: 6631: fatty acid | 244 | 1.429 | 15 | 3.289 | 0.00243 |
| metabolism | | | | | |
| GO: 45428: regulation of | 22 | 0.129 | 4 | 0.877 | 0.00251 |
| nitric oxide biosynthesis | | | | | |
| GO: 186: activation of | 22 | 0.129 | 4 | 0.877 | 0.00251 |
| MAPKK activity | | | | | |
| GO: 9605: response to | 1153 | 6.755 | 47 | 10.31 | 0.00252 |
| external stimulus | | | | | |
| GO: 48637: skeletal | 89 | 0.521 | 8 | 1.754 | 0.0026 |
| muscle development | | | | | |
| GO: 2011: morphogenesis | 11 | 0.0644 | 3 | 0.658 | 0.00266 |
| of an epithelial sheet | | | | | |
| GO: 30097: hemopoiesis | 298 | 1.746 | 17 | 3.728 | 0.00283 |
| GO: 80: G1 phase of | 37 | 0.217 | 5 | 1.096 | 0.00287 |
| mitotic cell cycle | | | | | |
| GO: 30316: osteoclast | 23 | 0.135 | 4 | 0.877 | 0.00297 |
| differentiation | | | | | |
| GO: 7165: signal | 4308 | 25.24 | 141 | 30.92 | 0.00321 |
| transduction | | | | | |
| GO: 6118: electron | 434 | 2.543 | 22 | 4.825 | 0.00322 |
| transport | | | | | |
| GO: 9613: response to | 778 | 4.558 | 34 | 7.456 | 0.00343 |
| pest, pathogen or parasite | | | | | |
| GO: 43118: negative | 1613 | 9.45 | 61 | 13.38 | 0.00344 |
| regulation of | | | | | |
| physiological process | | | | | |
| GO: 50874: organismal | 3071 | 17.99 | 105 | 23.03 | 0.00345 |
| physiological process | | | | | |
| GO: 6955: immune | 1298 | 7.604 | 51 | 11.18 | 0.00353 |
| response | | | | | |
| GO: 50896: response to | 3151 | 18.46 | 107 | 23.46 | 0.00389 |
| stimulus | | | | | |
| GO: 45859: regulation of | 283 | 1.658 | 16 | 3.509 | 0.00405 |
| protein kinase activity | | | | | |
| GO: 16572: histone | 4 | 0.0234 | 2 | 0.439 | 0.00412 |
| phosphorylation | | | | | |
| GO: 9441: glycolate | 4 | 0.0234 | 2 | 0.439 | 0.00412 |
| metabolism | | | | | |
| GO: 42752: regulation of | 4 | 0.0234 | 2 | 0.439 | 0.00412 |
| circadian rhythm | | | | | |
| GO: 51338: regulation of | 284 | 1.664 | 16 | 3.509 | 0.00419 |
| transferase activity | | | | | |
| GO: 8015: circulation | 235 | 1.377 | 14 | 3.07 | 0.00441 |
| GO: 6379: mRNA | 13 | 0.0762 | 3 | 0.658 | 0.00444 |
| cleavage | | | | | |
| GO: 45655: regulation of | 26 | 0.152 | 4 | 0.877 | 0.00471 |
| monocyte differentiation | | | | | |
| GO: 42417: dopamine | 26 | 0.152 | 4 | 0.877 | 0.00471 |
| metabolism | | | | | |
| GO: 45786: negative | 367 | 2.15 | 19 | 4.167 | 0.00478 |
| regulation of progression | | | | | |
| through cell cycle | | | | | |
| GO: 48534: hemopoietic | 314 | 1.84 | 17 | 3.728 | 0.00479 |
| or lymphoid organ | | | | | |
| development | | | | | |
| GO: 51243: negative | 1574 | 9.221 | 59 | 12.94 | 0.00485 |
| regulation of cellular | | | | | |
| physiological process | | | | | |
| GO: 45595: regulation of | 238 | 1.394 | 14 | 3.07 | 0.00493 |
| cell differentiation | | | | | |
| GO: 8277: regulation of | 60 | 0.352 | 6 | 1.316 | 0.00521 |
| G-protein coupled | | | | | |
| receptor protein signaling | | | | | |
| pathway | | | | | |
| GO: 6357: regulation of | 775 | 4.54 | 33 | 7.237 | 0.00576 |
| transcription from RNA | | | | | |
| polymerase II promoter | | | | | |
| GO: 1525: angiogenesis | 218 | 1.277 | 13 | 2.851 | 0.00592 |
| GO: 43207: response to | 812 | 4.757 | 34 | 7.456 | 0.00655 |
| external biotic stimulus | | | | | |
| GO: 45639: positive | 45 | 0.264 | 5 | 1.096 | 0.00675 |
| regulation of myeloid cell | | | | | |
| differentiation | | | | | |
| GO: 51260: protein | 45 | 0.264 | 5 | 1.096 | 0.00675 |
| homooligomerization | | | | | |
| GO: 51318: G1 phase | 45 | 0.264 | 5 | 1.096 | 0.00675 |
| GO: 30216: keratinocyte | 47 | 0.275 | 5 | 1.096 | 0.00812 |
| differentiation | | | | | |
| GO: 42491: auditory | 16 | 0.0937 | 3 | 0.658 | 0.00819 |
| receptor cell | | | | | |
| differentiation | | | | | |
| GO: 42135: | 16 | 0.0937 | 3 | 0.658 | 0.00819 |
| neurotransmitter | | | | | |
| catabolism | | | | | |
| GO: 7169: transmembrane | 334 | 1.957 | 17 | 3.728 | 0.00867 |
| receptor protein tyrosine | | | | | |
| kinase signaling pathway | | | | | |
| GO: 6952: defense | 1394 | 8.167 | 52 | 11.4 | 0.00884 |
| response | | | | | |
| GO: 48730: epidermis | 48 | 0.281 | 5 | 1.096 | 0.00887 |
| morphogenesis | | | | | |
| GO: 1568: blood vessel | 283 | 1.658 | 15 | 3.289 | 0.00936 |
| development | | | | | |
| GO: 42221: response to | 623 | 3.65 | 27 | 5.921 | 0.00959 |
| chemical stimulus | | | | | |
| GO: 45446: endothelial | 17 | 0.0996 | 3 | 0.658 | 0.00975 |
| cell differentiation | | | | | |
| GO: 48009: insulin-like | 17 | 0.0996 | 3 | 0.658 | 0.00975 |
| growth factor receptor | | | | | |
| signaling pathway | | | | | |
| GO: 9891: positive | 90 | 0.527 | 7 | 1.535 | 0.0103 |
| regulation of biosynthesis | | | | | |
| GO: 1944: vasculature | 288 | 1.687 | 15 | 3.289 | 0.0109 |
| development | | | | | |
| GO: 8286: insulin receptor | 70 | 0.41 | 6 | 1.316 | 0.0109 |
| signaling pathway | | | | | |
| GO: 6366: transcription | 1094 | 6.409 | 42 | 9.211 | 0.0115 |
| from RNA polymerase II | | | | | |
| promoter | | | | | |
| GO: 50789: regulation of | 5971 | 34.98 | 183 | 40.13 | 0.0116 |
| biological process | | | | | |
| GO: 43122: regulation of | 162 | 0.949 | 10 | 2.193 | 0.0118 |
| I-kappaB kinase/NF- | | | | | |
| kappaB cascade | | | | | |
| GO: 7500: mesodermal | 7 | 0.041 | 2 | 0.439 | 0.0137 |
| cell fate determination | | | | | |
| GO: 45672: positive | 7 | 0.041 | 2 | 0.439 | 0.0137 |
| regulation of osteoclast | | | | | |
| differentiation | | | | | |
| GO: 42448: progesterone | 7 | 0.041 | 2 | 0.439 | 0.0137 |
| metabolism | | | | | |
| GO: 17145: stem cell | 7 | 0.041 | 2 | 0.439 | 0.0137 |
| division | | | | | |
| GO: 50847: progesterone | 7 | 0.041 | 2 | 0.439 | 0.0137 |
| receptor signaling | | | | | |
| pathway | | | | | |
| GO: 50791: regulation of | 5273 | 30.89 | 163 | 35.75 | 0.0139 |
| physiological process | | | | | |
| GO: 1822: kidney | 54 | 0.316 | 5 | 1.096 | 0.0144 |
| development | | | | | |
| GO: 2009: morphogenesis | 143 | 0.838 | 9 | 1.974 | 0.0147 |
| of an epithelium | | | | | |
| GO: 7160: cell-matrix | 143 | 0.838 | 9 | 1.974 | 0.0147 |
| adhesion | | | | | |
| GO: 48514: blood vessel | 245 | 1.435 | 13 | 2.851 | 0.0148 |
| morphogenesis | | | | | |
| GO: 42330: taxis | 193 | 1.131 | 11 | 2.412 | 0.0149 |
| GO: 6935: chemotaxis | 193 | 1.131 | 11 | 2.412 | 0.0149 |
| GO: 35315: hair cell | 20 | 0.117 | 3 | 0.658 | 0.0154 |
| differentiation | | | | | |
| GO: 42133: | 55 | 0.322 | 5 | 1.096 | 0.0155 |
| neurotransmitter | | | | | |
| metabolism | | | | | |
| GO: 7166: cell surface | 1904 | 11.15 | 66 | 14.47 | 0.016 |
| receptor linked signal | | | | | |
| transduction | | | | | |
| GO: 48469: cell | 145 | 0.849 | 9 | 1.974 | 0.016 |
| maturation | | | | | |
| GO: 31589: cell-substrate | 145 | 0.849 | 9 | 1.974 | 0.016 |
| adhesion | | | | | |
| GO: 7243: protein kinase | 591 | 3.462 | 25 | 5.482 | 0.0163 |
| cascade | | | | | |
| GO: 9913: epidermal cell | 37 | 0.217 | 4 | 0.877 | 0.0166 |
| differentiation | | | | | |
| GO: 9887: organ | 868 | 5.085 | 34 | 7.456 | 0.0167 |
| morphogenesis | | | | | |
| GO: 7219: Notch | 77 | 0.451 | 6 | 1.316 | 0.0169 |
| signaling pathway | | | | | |
| GO: 9967: positive | 223 | 1.306 | 12 | 2.632 | 0.017 |
| regulation of signal | | | | | |
| transduction | | | | | |
| GO: 7242: intracellular | 1845 | 10.81 | 64 | 14.04 | 0.0174 |
| signaling cascade | | | | | |
| GO: 9607: response to | 1448 | 8.483 | 52 | 11.4 | 0.0174 |
| biotic stimulus | | | | | |
| GO: 7167: enzyme linked | 476 | 2.789 | 21 | 4.605 | 0.0175 |
| receptor protein signaling | | | | | |
| pathway | | | | | |
| GO: 6629: lipid | 935 | 5.478 | 36 | 7.895 | 0.0178 |
| metabolism | | | | | |
| GO: 48333: mesodermal | 8 | 0.0469 | 2 | 0.439 | 0.0179 |
| cell differentiation | | | | | |
| GO: 1710: mesodermal | 8 | 0.0469 | 2 | 0.439 | 0.0179 |
| cell fate commitment | | | | | |
| GO: 45657: positive | 8 | 0.0469 | 2 | 0.439 | 0.0179 |
| regulation of monocyte | | | | | |
| differentiation | | | | | |
| GO: 42420: dopamine | 8 | 0.0469 | 2 | 0.439 | 0.0179 |
| catabolism | | | | | |
| GO: 42424: | 8 | 0.0469 | 2 | 0.439 | 0.0179 |
| catecholamine catabolism | | | | | |
| GO: 42572: retinol | 8 | 0.0469 | 2 | 0.439 | 0.0179 |
| metabolism | | | | | |
| GO: 48512: circadian | 8 | 0.0469 | 2 | 0.439 | 0.0179 |
| behavior | | | | | |
| GO: 42745: circadian | 8 | 0.0469 | 2 | 0.439 | 0.0179 |
| sleep/wake cycle | | | | | |
| GO: 43124: negative | 8 | 0.0469 | 2 | 0.439 | 0.0179 |
| regulation of I-kappaB | | | | | |
| kinase/NF-kappaB | | | | | |
| cascade | | | | | |
| GO: 7050: cell cycle | 148 | 0.867 | 9 | 1.974 | 0.018 |
| arrest | | | | | |
| GO: 48332: mesoderm | 38 | 0.223 | 4 | 0.877 | 0.0181 |
| morphogenesis | | | | | |
| GO: 902: cellular | 720 | 4.218 | 29 | 6.36 | 0.0186 |
| morphogenesis | | | | | |
| GO: 1657: ureteric bud | 40 | 0.234 | 4 | 0.877 | 0.0215 |
| development | | | | | |
| GO: 6584: catecholamine | 40 | 0.234 | 4 | 0.877 | 0.0215 |
| metabolism | | | | | |
| GO: 46209: nitric oxide | 40 | 0.234 | 4 | 0.877 | 0.0215 |
| metabolism | | | | | |
| GO: 6809: nitric oxide | 40 | 0.234 | 4 | 0.877 | 0.0215 |
| biosynthesis | | | | | |
| GO: 45445: myoblast | 60 | 0.352 | 5 | 1.096 | 0.0218 |
| differentiation | | | | | |
| GO: 51239: regulation of | 371 | 2.174 | 17 | 3.728 | 0.0222 |
| organismal physiological | | | | | |
| process | | | | | |
| GO: 30431: sleep | 9 | 0.0527 | 2 | 0.439 | 0.0226 |
| GO: 9611: response to | 672 | 3.937 | 27 | 5.921 | 0.0233 |
| wounding | | | | | |
| GO: 1655: urogenital | 61 | 0.357 | 5 | 1.096 | 0.0233 |
| system development | | | | | |
| GO: 18958: phenol | 41 | 0.24 | 4 | 0.877 | 0.0234 |
| metabolism | | | | | |
| GO: 7249: I-kappaB | 207 | 1.213 | 11 | 2.412 | 0.0236 |
| kinase/NF-kappaB | | | | | |
| cascade | | | | | |
| GO: 51348: negative | 84 | 0.492 | 6 | 1.316 | 0.0249 |
| regulation of transferase | | | | | |
| activity | | | | | |
| GO: 6469: negative | 84 | 0.492 | 6 | 1.316 | 0.0249 |
| regulation of protein | | | | | |
| kinase activity | | | | | |
| GO: 9190: cyclic | 42 | 0.246 | 4 | 0.877 | 0.0253 |
| nucleotide biosynthesis | | | | | |
| GO: 42490: | 24 | 0.141 | 3 | 0.658 | 0.0253 |
| mechanoreceptor | | | | | |
| differentiation | | | | | |
| GO: 6950: response to | 1752 | 10.26 | 60 | 13.16 | 0.0265 |
| stress | | | | | |
| GO: 42078: germ-line | 1 | 0.00586 | 1 | 0.219 | 0.0267 |
| stem cell division | | | | | |
| GO: 48133: male germ- | 1 | 0.00586 | 1 | 0.219 | 0.0267 |
| line stem cell division | | | | | |
| GO: 48319: axial | 1 | 0.00586 | 1 | 0.219 | 0.0267 |
| mesoderm morphogenesis | | | | | |
| GO: 50872: white fat cell | 1 | 0.00586 | 1 | 0.219 | 0.0267 |
| differentiation | | | | | |
| GO: 7423: sensory organ | 1 | 0.00586 | 1 | 0.219 | 0.0267 |
| development | | | | | |
| GO: 46439: L-cysteine | 1 | 0.00586 | 1 | 0.219 | 0.0267 |
| metabolism | | | | | |
| GO: 6701: progesterone | 1 | 0.00586 | 1 | 0.219 | 0.0267 |
| biosynthesis | | | | | |
| GO: 48178: negative | 1 | 0.00586 | 1 | 0.219 | 0.0267 |
| regulation of hepatocyte | | | | | |
| growth factor | | | | | |
| biosynthesis | | | | | |
| GO: 48176: regulation of | 1 | 0.00586 | 1 | 0.219 | 0.0267 |
| hepatocyte growth factor | | | | | |
| biosynthesis | | | | | |
| GO: 48175: hepatocyte | 1 | 0.00586 | 1 | 0.219 | 0.0267 |
| growth factor | | | | | |
| biosynthesis | | | | | |
| GO: 42362: fat-soluble | 1 | 0.00586 | 1 | 0.219 | 0.0267 |
| vitamin biosynthesis | | | | | |
| GO: 35238: vitamin A | 1 | 0.00586 | 1 | 0.219 | 0.0267 |
| biosynthesis | | | | | |
| GO: 42904: 9-cis-retinoic | 1 | 0.00586 | 1 | 0.219 | 0.0267 |
| acid biosynthesis | | | | | |
| GO: 42412: taurine | 1 | 0.00586 | 1 | 0.219 | 0.0267 |
| biosynthesis | | | | | |
| GO: 46022: positive | 1 | 0.00586 | 1 | 0.219 | 0.0267 |
| regulation of transcription | | | | | |
| from RNA polymerase II | | | | | |
| promoter, mitotic | | | | | |
| GO: 46021: regulation of | 1 | 0.00586 | 1 | 0.219 | 0.0267 |
| transcription from RNA | | | | | |
| polymerase II promoter, | | | | | |
| mitotic | | | | | |
| GO: 45896: regulation of | 1 | 0.00586 | 1 | 0.219 | 0.0267 |
| transcription, mitotic | | | | | |
| GO: 45897: positive | 1 | 0.00586 | 1 | 0.219 | 0.0267 |
| regulation of | | | | | |
| transcription, mitotic | | | | | |
| GO: 19530: taurine | 1 | 0.00586 | 1 | 0.219 | 0.0267 |
| metabolism | | | | | |
| GO: 42905: 9-cis-retinoic | 1 | 0.00586 | 1 | 0.219 | 0.0267 |
| acid metabolism | | | | | |
| GO: 1887: selenium | 1 | 0.00586 | 1 | 0.219 | 0.0267 |
| metabolism | | | | | |
| GO: 50783: cocaine | 1 | 0.00586 | 1 | 0.219 | 0.0267 |
| metabolism | | | | | |
| GO: 8633: activation of | 1 | 0.00586 | 1 | 0.219 | 0.0267 |
| pro-apoptotic gene | | | | | |
| products | | | | | |
| GO: 45746: negative | 1 | 0.00586 | 1 | 0.219 | 0.0267 |
| regulation of Notch | | | | | |
| signaling pathway | | | | | |
| GO: 50794: regulation of | 5521 | 32.35 | 167 | 36.62 | 0.0278 |
| cellular process | | | | | |
| GO: 31269: | 10 | 0.0586 | 2 | 0.439 | 0.0278 |
| pseudopodium formation | | | | | |
| GO: 31272: regulation of | 10 | 0.0586 | 2 | 0.439 | 0.0278 |
| pseudopodium formation | | | | | |
| GO: 31274: positive | 10 | 0.0586 | 2 | 0.439 | 0.0278 |
| regulation of | | | | | |
| pseudopodium formation | | | | | |
| GO: 31268: | 10 | 0.0586 | 2 | 0.439 | 0.0278 |
| pseudopodium | | | | | |
| organization and | | | | | |
| biogenesis | | | | | |
| GO: 7622: rhythmic | 10 | 0.0586 | 2 | 0.439 | 0.0278 |
| behavior | | | | | |
| GO: 30278: regulation of | 25 | 0.146 | 3 | 0.658 | 0.0282 |
| ossification | | | | | |
| GO: 7528: neuromuscular | 25 | 0.146 | 3 | 0.658 | 0.0282 |
| junction development | | | | | |
| GO: 6979: response to | 87 | 0.51 | 6 | 1.316 | 0.0289 |
| oxidative stress | | | | | |
| GO: 8154: actin | 111 | 0.65 | 7 | 1.535 | 0.0293 |
| polymerization and/or | | | | | |
| depolymerization | | | | | |
| GO: 30224: monocyte | 44 | 0.258 | 4 | 0.877 | 0.0294 |
| differentiation | | | | | |
| GO: 7422: peripheral | 26 | 0.152 | 3 | 0.658 | 0.0312 |
| nervous system | | | | | |
| development | | | | | |
| GO: 30178: negative | 26 | 0.152 | 3 | 0.658 | 0.0312 |
| regulation of Wnt | | | | | |
| receptor signaling | | | | | |
| pathway | | | | | |
| GO: 8284: positive | 332 | 1.945 | 15 | 3.289 | 0.0339 |
| regulation of cell | | | | | |
| proliferation | | | | | |
| GO: 1656: metanephros | 46 | 0.269 | 4 | 0.877 | 0.034 |
| development | | | | | |
| GO: 46850: regulation of | 27 | 0.158 | 3 | 0.658 | 0.0345 |
| bone remodeling | | | | | |
| GO: 51259: protein | 91 | 0.533 | 6 | 1.316 | 0.035 |
| oligomerization | | | | | |
| GO: 7049: cell cycle | 1384 | 8.108 | 48 | 10.53 | 0.0373 |
| GO: 6171: cAMP | 28 | 0.164 | 3 | 0.658 | 0.0379 |
| biosynthesis | | | | | |
| GO: 19752: carboxylic | 736 | 4.312 | 28 | 6.14 | 0.0387 |
| acid metabolism | | | | | |
| GO: 30855: epithelial cell | 70 | 0.41 | 5 | 1.096 | 0.0391 |
| differentiation | | | | | |
| GO: 31346: positive | 12 | 0.0703 | 2 | 0.439 | 0.0394 |
| regulation of cell | | | | | |
| projection organization | | | | | |
| and biogenesis | | | | | |
| GO: 48731: system | 1158 | 6.784 | 41 | 8.991 | 0.0396 |
| development | | | | | |
| GO: 6082: organic acid | 738 | 4.324 | 28 | 6.14 | 0.0398 |
| metabolism | | | | | |
| GO: 17148: negative | 29 | 0.17 | 3 | 0.658 | 0.0414 |
| regulation of protein | | | | | |
| biosynthesis | | | | | |
| GO: 9628: response to | 775 | 4.54 | 29 | 6.36 | 0.0428 |
| abiotic stimulus | | | | | |
| GO: 6959: humoral | 258 | 1.512 | 12 | 2.632 | 0.045 |
| immune response | | | | | |
| GO: 302: response to | 30 | 0.176 | 3 | 0.658 | 0.0451 |
| reactive oxygen species | | | | | |
| GO: 45087: innate | 73 | 0.428 | 5 | 1.096 | 0.0455 |
| immune response | | | | | |
| GO: 46627: negative | 13 | 0.0762 | 2 | 0.439 | 0.0457 |
| regulation of insulin | | | | | |
| receptor signaling | | | | | |
| pathway | | | | | |
| GO: 30041: actin filament | 51 | 0.299 | 4 | 0.877 | 0.0469 |
| polymerization | | | | | |
| GO: 7519: striated muscle | 150 | 0.879 | 8 | 1.754 | 0.0484 |
| development | | | | | |

### REFERENCES

1. E. Alsberg, K. W. Anderson, A. Albeiruti, J. A. Rowley, and D. J. Mooney, Engineering growing tissues, Proc Natl Acad Sci U.S.A 99:12025 (2002).
2. N. G. Genes, J. A. Rowley, D. J. Mooney, and L. J. Bonassar, Effect of substrate mechanics on chondrocyte adhesion to modified alginate surfaces, Archives of Biochemsitry and Biophysics 422:161 (2004).
3. J. E. Grimmer, C. B. Gunnlaugsson, E. Alsberg, H. S. Murphy, H.-J. Kong, D. J. Mooney, and R. A. Weatherly, Tracheal reconstruction using tissue-engineered cartilage, Arch. Otolaryngol. Head Neck Surrg. 130:1191 (2004).
4. P. K. Kreeger, J. W. Deck, T. K. Woodruff, and L. D. Shea, The in vitro regulation of ovarian follicle development using alginate-extracellular matrix gels, Biomaterials 27:714 (2006).
5. W. F. Liu and C. S. Chen, Engineering biomaterials to control cell function, Materials Today 8:28 (2005).
6. A. Loebsack, K. Greene, S. Wyatt, C. Culberson, C. Austin, R. Beiler, W. Roland, P. Eiselt, J. A. Rowley, K. Burg, D. J. Mooney, W. Holder, and C. Halberstadt, In vivo characterization of a porous hydrogel material for use as a tissue bulking agent, Journal of Biomedical Materials Research 57:575 (2001).
7. J. J. Marler, A. Guha, J. Rowley, R. Koka, D. J. Mooney, J. Upton, and J. P. Vacanti, Soft-tissue augmentation with injectable alginate and syngeneic fibroblasts, Plastic.and reconstructive.surgery 105:2049 (2000).
8. D. J. Mooney, K. H. Bouhadir, W. K. Wong, and J. A. Rowley. Polymers containing polysaccharides such as alginates or modified alginates. The Regents of the University of Michigan. patent Ser. No. 09/147,900[U.S. Pat. No. 6,642,363]. 2003. MI/USA.
9. J. A. Rowley, G. Madlambayan, and D. J. Mooney, Alginate hydrogels as synthetic extracellular matrix materials, Biomaterials 20:45 (1999).
10. J. A. Rowley and D. J. Mooney, Alginate type and RGD density control myoblast phenotype, Journal of Biomedical Materials Research 60:217 (2003).
11. E. Ruoslahti and R. Pasqualini. Structural mimics of RGD-binding sites. U.S. Pat. No. 5,817,750. 1998. USA.
12. Caplan A I. Mesenchymal stem cells. J Orthop Res 1991; 9:641-650.
13. Pittenger M F, Mackay A M, Beck S C et al. Multilineage potential of adult human mesenchymal stem cells. Science 1999; 284:143-147.
14. Zuk P A, Zhu M, Ashjian P et al. Human adipose tissue is a source of multipotent stem cells. Mol Biol Cell 2002; 13:4279-4295.
15. Lakshmipathy U, Verfaillie C. Stem cell plasticity. Blood Rev 2005; 19:29-38.
16. Dominici M, Le B K, Mueller I et al. Minimal criteria for defining multipotent mesenchymal stromal cells. The International Society for Cellular Therapy position statement. Cytotherapy 2006; 8:315-317.
17. Yamada K M, Cukierman E. Modeling tissue morphogenesis and cancer in 3D. Cell 2007; 130:601-610.
18. Boquest A C, Shandadfar A, Fronsdal K et al. Isolation and transcription profiling of purified uncultured human stromal stem cells: alteration of gene expression after in vitro cell culture. Mol Biol Cell 2005; 16:1131-1141.
19. Gentleman R C, Carey V J, Bates D M et al. Bioconductor: open software development for computational biology and bioinformatics. Genome Biol 2004; 5:R80-
20. Wu Z, Irizarry R A, Gentleman R et al. A Model-Based Background Adjustment for Oligonucleotide Expression Arrays. Journal of the American Statistical Association 2004; 99:909-
21. Smyth G K. Linear models and empirical Bayes methods for assessing differential expression in microarray experiments. Statistical Applications in Genetics and Molecular Biology 2004; 3:
22. Gautier L, Cope L, Bolstad B M et al. affy—analysis of Affymetrix GeneChip data at the probe level. Bioinformatics 2004; 20:307-315.
23. Benjamini Y, Hochberg Y. Controlling the false discovery rate: a practical and powerful approach to multiple testing. Journal of the Royal Statistical Society 1995; 57:
24. Frisch S M, Ruoslahti E. Integrins and anoikis. Curr Opin Cell Biol 1997; 9:701-706.
25. Boquest A C, Shandadfar A, Brinchmann J E et al. Isolation of stromal stem cells from human adipose tissue. Methods Mol Biol 2006; 325:35-46.
26. Roth S, Neuman-Silberberg F S, Barcelo G et al. cornichon and the EGF receptor signaling process are necessary for both anterior-posterior and dorsal-ventral pattern formation in Drosophila. Cell 1995; 81:967-978.
27. Takikawa O. Biochemical and medical aspects of the indoleamine 2,3-dioxygenase-initiated L-tryptophan metabolism. Biochem Biophys Res Commun 2005; 338:12-19.
28. Meisel R, Zibert A, Laryea M et al. Human bone marrow stromal cells inhibit allogeneic T-cell responses by indoleamine 2,3-dioxygenase-mediated tryptophan degradation. Blood 2004; 103:4619-4621.
29. Arnaout M A, Goodman S L, Xiong J P. Coming to grips with integrin binding to ligands. Curr Opin Cell Biol 2002; 14:641-651.
30. Gilmore A P. Anoikis. Cell Death Differ 2005; 12 Suppl 2:1473-1477.
31. Markusen J F, Mason C, Hull D A et al. Behavior of adult human mesenchymal stem cells entrapped in alginate-GRGDY beads. Tissue Eng 2006; 12:821-830.
32. Schnabel M, Marlovits S, Eckhoff G et al. Dedifferentiation-associated changes in morphology and gene expression in primary human articular chondrocytes in cell culture. Osteoarthritis Cartilage 2002; 10:62-70.

1-11. (canceled)

12. A method of treating an individual who has an injury involving nerve cells or a degenerative disease comprising the step of administering a plurality of stem cells to said individual in an amount effective and at a site effective to provide a therapeutic benefit to the individual; wherein said plurality of stem cells are prepared by a process comprising (i) maintaining said stem cells in a monolayer, (ii) subsequently entrapping said stem cells in a biostructure, wherein the biostructure comprises a modified alginate that comprises at least one alginate chain section to which is bonded by covalent bonding at least one cell attachment peptide and said modified alginate comprises no more than 500 EU/g of endotoxin, and (iii) isolating said stem cells from said biostructure.

13. The method of claim 12 wherein the individual has an injury involving nerve damage.

14. The method of claim 12 wherein the individual has a neurological disorder.

15. The method of claim 12 wherein the individual has a degenerative disease selected from the group consisting of Alzheimer's Disease; Amyotrophic Lateral Sclerosis, i.e., Lou Gehrig's Disease; Atherosclerosis; Cancer; Diabetes, Heart Disease; Huntington's disease; Inflammatory Bowel Disease; mucopolysaccharidosis; Multiple Sclerosis; Norrie disease; Parkinson's Disease; Prostatitis; Osteoarthritis; Osteoporosis; Shy-Drager syndrome; and Stroke.

16. The method of claim 12, wherein said biostructure is a gel, foam, bead, scaffold, fibre, felt, sponge or combinations thereof.

17. The method of claim 12, wherein said cell attachment peptide contains one or more RGD sequences.

18. The method of claim 12, wherein said stem cells are mesenchymal stem cells.

19. The method of claim 12, wherein said stem cells are isolated from said biostructure by adding at least one cation binding agent to said biostructure.

20. The method of claim 12, wherein said injury involves nerve cells.

21. The method of claim 15, wherein said disease is selected from the group consisting of Amyotrophic Lateral Sclerosis, Multiple sclerosis and Parkinson's disease.

22. The method of claim 12, wherein said administration is systemic.

23. The method of claim 12, wherein said administration is by injection.

24. The method of claim 12, wherein said administration is delivered intravenously, intrathecally or subcutaneously.

25. The method of the previous claim, wherein said administration is delivered to a spinal column or nerve pathway.
